(19) United States
(12) Patent Application Publication
(10) Pub. No.: US 2003/0113910 A1
(43) Pub. Date: Jun. 19, 2003

(54) Pluripotent stem cells derived without the use of embryos or fetal tissue

(76) Inventor: Mike Levanduski, River Vale, NJ (US)

Correspondence Address: DAVIDSON, DAVIDSON & KAPPEL, LLC, 14th Floor, 485 Seventh Avenue, New York, NY, 10018, US

(21) Appl. No.: 10/026,420

(22) Filed: Dec. 19, 2001

(51) Int. Cl. C12N 5/06; C12N 5/08; C12N 15/85

(52) U.S. Cl. 435/325; 435/354; 435/366

(57) ABSTRACT

This invention provides a method for deriving precursors to pluripotent non-embryonic stem (P-PNES) and pluripotent non-embryonic stem (PNES) cell lines. The present invention involves nuclear transfer of genetic material from a somatic cell into an enucleated, zona pellucida free human ooplastoid having a reduced amount of total cytoplasm. The present invention provides a new source for obtaining human and other animal pluripotent stem cells. The source utilizes as starting materials an oocyte and a somatic cell as the starting materials but does not require the use, creation and/or destruction of embryos or fetal tissue and does not in any way involve creating a cloned being. The oocyte never becomes fertilized and never develops into an embryo. Rather, portions of the oocyte cytoplasm are extracted and combined with the nuclear material of individual mature somatic cells in a manner that precludes embryo formation. Murine, bovine, and human examples of the procedure are demonstrated. Subsequently, the newly constructed P-PNES cells are cultured in vitro and give rise to PNES cells and cell colonies. Methods are described for culturing the P-PNES cells to yield purified PNES cells which have the ability to differentiate into cells derived from mesoderm, endoderm, and ectoderm germ layers. Methods are described for maintaining and proliferating PNES cells in culture in an undifferentiated state. Methods and results are described for analysis and validation of pluripotency of PNES cells including cell morphology, cell surface markers, pluripotent tumor development in SCID mouse, karyotyping, immortality in in vitro culture.

### TECHNICAL FIELD OF THE INVENTION

This invention relates to the creation, production, maintenance, growth and application of human and animal pluripotent stem cells that have been created without the use and/or destruction of embryos (whether naturally derived or created via a cloning process) and without the need for fetal tissue, or “pluripotent non-embryonic/non-fetal tissue derived stem cells” (hereinafter, “PNES,” and reference to “PNES” throughout this filing shall incorporate both human and animal PNES cells unless otherwise indicated). More specifically this invention provides (a) a method for deriving cells which are precursors to PNES cells (“P-PNES cells”) via the nuclear transfer of genetic material from a somatic cell into an enucleated, zona pellucida free portion of an ooplast having a reduced amount of total ooplasm (referred to as an “ooplastoid”), and a method for keeping those P-PNES cells from clumping or gathering into a cell mass, (b) methods of culturing and converting the P-PNES cells into actual PNES cells and PNES cell lines and for methods/techniques for establishing the characteristics (including immortality and pluripotency) of those PNES cells, (c) methods for maintaining and proliferating the PNES cells and PNES cell lines in an undifferentiated state, (d) methods and techniques for directing those PNES cells to become multipotent/adult stem cells including, but not limited to, blood stem cells, neural stem cells, liver stem cells, and other stem cells and/or Specific Differentiated Cells, (e) methods for directing those multipotent/adult stem cells to become more specialized (differentiated) cells which no longer have the ability to differentiate, including, but not limited to, sertoli cells, endothelial cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. and (f) the use of those P-PNES, PNES, multipotent/adult stem cells, and Specific Differentiated Cells and derivatives thereof for scientific and therapeutic purposes. The scientific and therapeutic applications include, but are not limited to, use in (a) scientific discovery and research involving cellular development and genetic research, (b) drug development and discovery, (c) gene therapy, and (d) treatment of diseases and disorders including, but not limited to, (i) tissue/cellular regeneration and replacement therapies and applications, (ii) immune system disorders, (iii) blood disorders, (iv) cancer, and a variety of other diseases and disorders.

### BACKGROUND OF THE INVENTION

“Pluripotent stem cells” are undifferentiated cells that have the potential to divide in vitro for a long period of time (greater than one year) and have the unique ability to differentiate into (and therefore are a potential source for) cells derived from all three embryonic germ layers—endoderm, mesoderm and ectoderm. This ability to differentiate into all three embryonic germ layers is referred to as “pluripotency.” The significant scientific and therapeutic potential of these cells, particularly because of their pluripotent nature, is monumental, and includes, but is not limited to, use in (a) scientific discovery and research involving cellular development and genetic research, (b) drug development and discovery, (c) gene therapy, and (d) tissue/cellular regeneration and replacement therapies and applications. It is also important to note that pluripotent stem cells do not have the ability to become an embryo or complete human or animal organism. In other words, these cells can differentiate into every cell found in a mature animal or human, but not the animal or human itself.

To date, there have been created two categories of pluripotent stem cells. “Embryonic stem cells,” as defined by the scientific community, are pluripotent stem cells that are derived directly from an embryo (to date, these embryos have been obtained via a naturally fertilized egg or via cloning). “Embryonic germ” cells are pluripotent stem cells that are derived directly from the fetal tissue of aborted fetuses. For purposes of simplicity, embryonic stem cells and embryonic germ cells will be collectively referred to as “ES” cells unless otherwise indicated. There are also reports that cells with some characteristics of human pluripotent ES cells may be created using a combination of human cells and oocytes from other animal species. Each of these current methods for creating pluripotent ES cells is described in more detail here.

As mentioned, two techniques are employed to create ES via the destruction of viable embryos. The first method utilizing human embryos was under U.S. Pat. Nos. 5,843,780 and 6,200,806, pursuant to which the inventor, Dr. Thompson, first derived a human ES cell line from the inner cell mass of normal human embryos in the blastocyst stage (U.S. Pat. No. 6,200,806 and Thompson, J. A. et al. Science, 282:1145-7, 1998). The blastocyst is formed approximately five days after fertilization of an oocyte by a sperm cell. The blastocyst stage embryos were donated by couples undergoing in vitro fertilization therapy. The ES stem cells produced by Thompson could proliferate in vitro, in an undifferentiated state, for more than one year if they were grown on a fibroblast feeder layer. These cells retained the ability to differentiate into endoderm, mesoderm or ectoderm lineage cells over this time period, thus displaying the characteristic of pluripotency. As a result of Dr. Thompson's process/method, the human embryos were destroyed. The second method for creating pluripotent ES cells which also involves the destruction of embryos utilizes the technique of somatic cell nuclear transfer (SCNT) in a practice pursuant to which an embryo is created via cloning, and then destroyed in the process that obtained the pluripotent ES cells from that embryo. The potential of this technique was demonstrated by Campbell and Wilmut using farm animal species wherein individual animals were cloned (See U.S. Pat. Nos. 6,147,276 and 6,252,133). In this technique the nucleus is removed from a normal egg, thus removing the genetic material. Next, a donor diploid somatic cell is placed next to the enucleated egg and the two cells are fused. The fused cell has the potential to develop into a viable embryo which may theoretically then be sacrificed in order to remove that portion of the embryo containing the stem cell producing inner cell mass. The use of this method in humans would thus involve creating a cloned embryo autologous to the donor of the somatic cells followed by the destruction of the human embryo.

Pursuant to another reported method that may create pluripotent ES cells, the nucleus of a human cell is transplanted into an entire enucleated animal oocyte of a species different from the donor cell (referred to herein as animal stem cell nuclear transfer, or “ASCNT”). See U.S. Pat. application Ser. No. 20010012513 (2001). The resultant chimeric cells are potentially used for the production of pluripotent ES cells, in particular human-like pluripotent ES cells. One disadvantage of this technique is that these chimeric cells may contain unknown non-human viruses and still contain the mitochondria of the animal species and thus there would be substantial risks of immune rejections if such cells were used in cell transplantation therapies.

The final reported technique for obtaining pluripotent ES cells requires the dissection of 8-11 week old aborted human fetuses. Under this method, human primordial embryonic germ cells are extracted from the gonadal ridges and mesenteries of aborted fetuses (U.S Pat. No. 6,090,622 and M. J. Shamblott et al. Proc. Natl. Acad. Sci. USA, 95:13726-13731, 1998). The human pluripotent ES cells produced in this manner were dependent on the presence of certain growth factors and ligands in the culture medium such as leukemia inhibitory factor (LIF), basic fibroblast growth factor and forskolin. In addition, the ES cells derived from human primordial embryonic germ cells differed slightly in cell morphology and surface marker expression from those derived from 5 day old blastocysts. These cells are believed to be pluripotent because immunohistochemical analysis of the embryoid bodies that form in cultures show antibody staining that is consistent with the presence of cells derived from the three embryonic germ layers.

Pluripotent stem cells (which include pluripotent ES cells) can be differentiated from “multipotent stem cells.” A multipotent stem cell has the ability to differentiate into some but not all of the cells derived from all three germ layers. For example, a “blood stem cell” is thought to be multipotent because it has the ability to differentiate into all types of specific blood cells, but it is unlikely that they can differentiate into all cells of a given animal or human. Multipotent stem cells exist in vivo (for example, blood stem cells can be found in bone marrow and the blood of adult animals and humans), and such in vivo cells also referred to as “adult stem cells.” In addition, multipotent stem cells can be created by directing pluripotent stem cells to become certain multipotent stem cells. (The term “multipotent/adult stem cell(s)” will be used to describe multipotent stem cells whether the source is in vivo or some other methodology or technique.) While not offering the same breadth of promise as pluripotent stem cells, multipotent/adult stem cells have a great deal of promise in research and in the area of therapeutic applications. For example, multipotent/adult stem cells have already been used in humans in attempts to treat certain blood, neural and cancer diseases.

It is also helpful to distinguish between pluripotent stem cells and “totitpotent stem cells.” Totipotent stem cells have the ability to not only differentiate into cells derived from all three germ layers just as pluripotent stem cells can, but they also have the ability to grow into a complete human being or animal, something which pluripotent stem cells such as pluripotent ES cells cannot accomplish.

Unfortunately, to date, pluripotent ES cells can only be derived from these sometimes—controversial sources—embryos (created naturally or via cloning), fetal tissue and via the mixing of materials of multiple species. The controversy surrounding the sources for such cells, according to many leading scientists and public and private organizations including the NIH, has greatly compromised and slowed the study of such cells and their application. In addition to the issues surrounding the sources of pluripotent ES cells, the other major shortcomings of some or all of the pluripotent ES cells created via current techniques include the following: (a) the use of current human ES lines obtained from the destruction of human embryos (e.g., those cell lines created by Dr. Thompson) is inappropriate according to the NIH because the cells have been exposed to animal cells (i.e., grown on mouse feeder layers); and (b) use of embryonic and fetal tissue derived stem cells may have limited application in humans because the genetic make-up of the resulting pluripotent ES cells will be different than that of any particular patient, causing issues of rejection by the immune system in the case, for example, of cellular or tissue transplants. Research and applications of multipotent/adult stem cells has also been hindered by various factors including (a) not all human adult stem cells have been isolated in tissue, (b) these cells are very difficult to isolate and purify, (c) they come in very minute quantities from in vivo sources and limited numbers are being created via the manipulation of pluripotent ES cells, (d) they do not last as long as pluripotent cells in vitro, (e) they are difficult to grow quickly enough to be used for acute disorders, (f) they can't be used to study early cell development, and (g) while they may be able to differentiate into other cells, they have not been shown to be pluripotent.

All of these major shortcomings have created a great demand for (a) methods of creating pluripotent ES cells without the use of embryos (naturally created or created via cloning) or fetal material and without the need to involve mixing of species cells or cell materials, (b) the ability to create pluripotent ES cells specific to a particular patient or disease population, a new and more plentiful and useful, and (c) a more plentiful source for multipotent/adult stem cells than is currently available.

### OBJECTS OF THE INVENTION

All of the objects set forth herein apply to humans and animals. “Animals” shall include ovine, bovines, porcine, equine, murine, and other laboratory, farm and/or household animals.

The objects of this invention include the following:

It is an object of the present invention to provide for a method for the creation of “ooplastoids,” which are enucleated, membraned, zona-pellucida free ooplasts and which result from the splitting of an enucleated oocyte into 2 to 6 portions.

It is an object of the present invention to provide ooplastoids.

It is an object of the present invention to provide a procedure for reprogramming a somatic cell nucleus using an “ooplastoid.”

It is an object of the present invention to provide a method for making ooplastoids that can be combined with somatic cells or somatic cell nuclei to give rise to precursors cells known as nascent cells which give rise to pluripotent non-embryonic/non-fetal tissue derived stem cells that are pluripotent and can proliferate in culture indefinitely and in an undifferentiated state. These precursor cells are referred to as “P-PNES” or “P-PNES cells.”

It is an object of the present invention to provide P-PNES cells.

It is an object of the present invention to provide P-PNES cells via nuclear transfer through combining an ooplastoid and a somatic cell or somatic cell nucleus.

It is a further object of the present invention to provide for a method for keeping P-PNES and PNES cells from clustering, grouping or contracting during in vitro culture.

It is further object of the present invention to culture and direct P-PNES cells into pluripotent non-embryonic/non-fetal tissue derived stem cells that are pluripotent and can proliferate in culture indefinitely and in an undifferentiated state (as indicated, these cells are referred to as “PNES” or “PNES cells” or “PNES cell lines”).

It is an object of this invention to provide P-PNES and PNES cells that can be identified, isolated and purified.

It is an object of this invention to provide for methods of identifying, isolating and purifying P-PNES cells and PNES cells.

It is a further object of this invention to provide PNES that can proliferate in culture in an undifferentiated state for more than one year and wherein the cells remain euploid..

It is another object of the present invention to provide for methods to maintain PNES cells in culture in an undifferentiated state.

It is a further object of the present invention to provide for methods of growing/proliferating PNES cells in culture.

It is an object of the present invention to provide PNES cells that retain the potential to differentiate into tissues derived from all three germ layers: endoderm, mesoderm, and ectoderm.

It is an object of the present invention to create P-PNES and PNES cells/cell lines that are not totipotent and are not embryogenic (e.g., human PNES cells can not develop into a human being if implanted in a woman's uterus).

It is an object of this invention to provide methods for creating P-PNES and PNES cells that are autologous to the source/donor of the somatic cell involved in the nuclear transfer and as a result it is the object of this invention to provide P-PNES and PNES cell lines that share the genetic make-up and characteristics of any specific/individual animal or human being or specific population (e.g. disease populations, racial populations, etc.).

It is an object of this invention to provide PNES cells that are autologous to the source/donor of the somatic cell involved in the nuclear transfer and as a result it is the object of this invention to provide P-PNES and PNES cell lines that share the genetic make-up and characteristics of any specific/individual animal or human being or specific population (e.g. disease populations, racial populations, etc.).

It is an object of the present invention to provide PNES cell lines which exhibit the same characteristics and properties of pluripotent ES cells (e.g., pluripotency, ability to remain undifferentiated in culture for more than one year, etc.), including characteristic and properties related to cell morphology, karyotypes, cell markers, and other tests/characteristics familiar to and accepted by the stem cell scientific community.

It is a further object of the current invention to provide for methods, tests and proofs utilized to prove the properties of PNES cells, including but not limited to tests to show/prove characteristics of pluripotency, cell morphology, karyotypes, and cell markers.

It is an object of this invention to provide for methods of utilizing PNES cells and their derivatives in scientific and therapeutic applications including, but not limited to, (a) scientific discovery and research involving cellular development and genetic research, (b) drug development and discovery (e.g., screening for efficacy and toxicity of certain drug candidates and chemicals), (c) gene therapy (e.g., as a delivery device for gene therapy), and (d) treatment of diseases and disorders including, but not limited to, Parkinson's, Alzehimer's, Huntington's, Ty Sachs, Gauchers, spinal cord injury, stoke, burns and other skin damage, heart disease, diabetes, Lupus, osteoarthritis, liver diseases, hormone disorders, kidney disease, leukemia, lymphoma, multiple sclerosis, rheumatoid arthritis, Duchenne's Musclar Dystrophy, Ontogenesis Imperfecto, birth defects, infertility, pregnancy loss, and other cancers, degenerative and other diseases and disorders.

It is a further object of this invention to provide for methods to direct PNES cells to differentiate into multipotent/adult stem cells derived from all three germ layers, including, but not exclusively, blood stem cells, neural stem cells, liver stem cells, and pancreatic stem cells.

It is a further object of this invention to provide/create multipotent/adult stem cells (derived from PNES) including, but not limited to, blood stem cells, neural stem cells, liver blood cells, and pancreatic stem cells.

It is a further object of this invention to provide for methods of identifying, isolating and purifying multipotent/adult stem cells derived from PNES, including, but not limited to, blood stem cells, neural stem cells, liver stem cells, and pancreatic stem cells.

It is a further object of this invention to provide for methods of proliferating multipotent/adult stem cells derived from PNES cells (including, but not limited to, blood stem cells, neural stem cells, liver stem cells, and pancreatic stem cells) in culture in an undifferentiated state.

It is a further object of the current invention to provide for methods, tests and proofs utilized to prove the properties of multipotent/adult stem cells derived from PNES, including but not limited to tests to show/prove characteristics of multipotency, cell morphology, karyotypes, and cell markers.

It is an object of this invention to provide for methods of utilizing multipotent/adult stem cells derived from PNES, and their derivatives, in scientific and therapeutic applications including, but not limited to, (a) scientific discovery and research involving cellular development and genetic research, (b) drug development and discovery (e.g., screening for efficacy and toxicity of certain drug candidates and chemicals), (c) gene therapy (e.g., as a delivery device for gene therapy), and (d) treatment of diseases and disorders including, but not limited to, Parkinson's, Alzehimer's, Huntington's, Ty Sachs, Gauchers, spinal cord injury, stoke, burns and other skin damage, heart disease, diabetes, Lupus, osteoarthritis, liver diseases, hormone disorders, kidney disease, leukemia, lymphoma, multiple sclerosis, rheumatoid arthritis, Duchenne's Musclar Dystrophy, Ontogenesis Imperfecto, birth defects, infertility, pregnancy loss, and other cancers, degenerative and other diseases and disorders.

It is a further object of this invention to provide for methods to direct multipotent/adult stem cells (derived from PNES cells) to differentiate into specific cell types derived from all three germ layers which have no capacity for further differentiation since they represent terminal differentiation stage (e.g., sertoli cells, endothelial cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. and hereinafter, referred to as “Specific Differentiated Cells”),

It is a further object of this invention to provide Specific Cell types which represent cells derived from all three germ layers and which do not have any differentiation abilities, including, but not limited to, sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc.

It is a further object of this invention to provide for methods of identifying, isolating and purifying Specific Differentiated Cells including, but not limited to, sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc.

It is a further object of this invention to provide for methods of proliferating Specific Differentiated Cells including, but not limited to sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc.

It is a further object of the current invention to provide for methods, tests and proofs utilized to prove the properties of Specific Differentiated Cells, including but not limited to tests to show/prove characteristics of cell morphology, karyotypes, and cell markers.

It is a further object of this invention to provide for methods of utilizing Specific Differentiated Cells and their derivatives in scientific and therapeutic applications including, but not limited to, (a) scientific discovery and research involving cellular development and genetic research, (b) drug development and discovery (e.g., screening for efficacy and toxicity of certain drug candidates and chemicals), (c) gene therapy (e.g., as a delivery device for gene therapy), and (d) treatment of diseases and disorders including, but not limited to, Parkinson's, Alzehimer's, Huntington's, Ty Sachs, Gauchers, spinal cord injury, stoke, burns and other skin damage, heart disease, diabetes, Lupus, osteoarthritis, liver diseases, hormone disorders, kidney disease, leukemia, lymphoma, multiple sclerosis, rheumatoid arthritis, Duchenne's Musclar Dystrophy, Ontogenesis Imperfecto, birth defects, infertility, pregnancy loss, and other cancers, degenerative and other diseases and disorders.

### SUMMARY OF THE INVENTION

The present invention provides a new source for obtaining pluripotent stem (PNES) cells. The process/method of creating PNES cells utilizes an oocyte and a somatic cell as the starting materials but does not require the use, creation and/or destruction of embryos or fetal tissue and does not in any way involve creating a cloned human or animal. This invention provides a method for deriving nascent cells which are precursors of PNES cells via nuclear transfer of genetic material from a somatic cell into an enucleated, zona pellucida free ooplast having a reduced amount of total cytoplasm. The oocyte used in this procedure never becomes fertilized and never develops into an embryo. Rather, portions of the oocyte cytoplasm are obtained and combined with the nuclear material of individual mature somatic cells in a manner that precludes embryo formation. Instead, the cells formed are precursors to PNES, or “P-PNES.” Subsequently, the newly constructed P-PNES cells are cultured in vitro and give rise to PNES cells and cell colonies. More specifically, this invention also provides (a) methods of isolating, identifying, and culturing the P-PNES cells to yield purified PNES cells which have the ability to differentiate into cells derived from mesoderm, endoderm, and ectoderm germ layers, (b) methods for isolating, purifying, identifying and maintaining and proliferating PNES cells in culture in an undifferentiated state for more than one year, and (c) the use of those PNES cells and derivatives thereof for scientific and therapeutic purposes. These applications include, but are not limited to, use of PNES cells and derivatives thereof in (a) scientific discovery and research involving cellular development and genetic research, (b) drug development and discovery, (c) gene therapy, and (d) tissue/cellular regeneration and replacement therapies and applications, and treatment for other diseases and disorders).

The current invention also provides for methods for directing pluripotent PNES cells to become multipotent/adult stem cells (referred to herein as ASC's) that individually have the ability to differentiate into some but not all of the cells derived from all three germ layers. For example, ASC's would include, but not exclusively, blood stem cells, which have the ability to differentiate into some, but not all, cells derived from all three germ layers. More specifically, this invention also provides (a) methods of culturing and directing PNES to yield purified ASC's which have the ability to differentiate into some but not all cells derived from mesoderm, endoderm, and ectoderm germ layers, (b) methods for isolating, purifying, identifying and maintaining and proliferating ASC's in culture in an undifferentiated state, and (c) the use of those ASC's and derivatives thereof for scientific and therapeutic purposes. These applications include, but are not limited to, use of ASC's and derivatives thereof in (a) scientific discovery and research involving cellular development and genetic research, (b) drug development and discovery, (c) gene therapy, and (d) tissue/cellular regeneration and replacement therapies and applications, and treatment for other diseases and disorders).

In addition to the above, the current invention provides for methods of directing ASC's to become Specific Differentiated Cells which no longer have the ability to differentiate, or “Specific Differentiated Cells” sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. More specifically, this invention also provides (a) methods of culturing and directing ASC's to yield purified Specific Differentiated Cells which no longer have the ability to differentiate, (b) the use of those Specific Differentiated Cells and derivatives thereof such as sertoli cells, endothelial cells, granulosa epithelial cells, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc for scientific and therapeutic purposes. These applications include, but are not limited to, use of Specific Differentiated Cells and derivatives thereof in (a) scientific discovery and research involving cellular development and genetic research, (b) drug development and discovery, (c) gene therapy, and (d) tissue/cellular regeneration and replacement therapies and applications, and treatment for other diseases and disorders).

In accordance of the above objects and others, the present invention is related in part to a purified preparation of pluripotent non-embryonic stem cells, which (i) is capable of proliferating in an in vitro culture for more than one year; (ii) maintains a karyotype in which the cells are euploid and are not altered through culture; (iii) maintains the potential to differentiate into cell types derived from the endoderm, mesoderm and ectoderm lineages throughout the culture, and (iv) is inhibited from differentiation when cultured on fibroblast feeder layers.

More particularly, the present invention is directed to pluripotent non-embryonic stem cells that display the following characteristics: the cells are negative for expression of the SSEA-1 marker; the cells express elevated alkaline phosphatase activity; the cells are positive for expression of the TRA-1-81 marker and the TRA-1-60 marker; the cells are positive for expression of the CCA-3 and CCA-4 Markers; and the cells are able to differentiate into cells derived from mesoderm, endoderm and ectoderm germ layers when the cells are injected into a SCID mouse.

This invention is further related to pluripotent non-embryonic stem cells and methods of producing them in which the cells are human, or non-human animal such as from the following animals: of dog, cat, mouse, rat, cow, pig, sheep, goat, horse, buffalo, llama, ferret, guinea pig, rabbit and any other mammalian species.

The invention is further related to a purified preparation of pluripotent non-embryonic stem cells, which (i) is capable of proliferating in an in vitro culture for an indefinite period; (ii) maintains a karyotype in which the cells are euploid and are not altered through culture; and (iii) maintains the potential to differentiate into cells types derived from the endoderm, mesoderm and ectoderm lineages throughout the culture.

The invention is further related to stem cells which do not originate from a fertilized egg, but which originates from the combination of a somatic cell nucleus and an enucleated ooplastoid.

The invention is further related to stem cells which do not originate from fetal tissue, but which originates from the combination of a somatic cell nucleus and an enucleated ooplastoid.

The present invention provides stem cells which do not originate from a fertilized egg or from fetal tissue, but which originates from the combination of a somatic cell nucleus and an enucleated ooplast or super-ooplast.

The invention is further related to stem cell which is produced by the method of (i) contacting a desired somatic cell or somatic cell nucleus with an ooplastoid, wherein the ooplastoid is derived from an enucleated oocyte; (ii) combining the somatic cell or somatic cell nucleus with an ooplastoid to create a nascent cell, and (iii) culturing the nascent cell to obtain pluripotent non-embryonic stem cells.

The invention is further related to a nascent cell produced from the combination of a somatic cell nucleus and an enucleated zona pellucida free ooplastoid.

In accordance with the above objects and others, the present invention provides method of producing pluripotent, non-embryonic stem cells comprising the following steps: (i) contacting a desired somatic cell or somatic cell nucleus with an ooplastoid, wherein the ooplastoid is derived from an enucleated oocyte; (ii) combining the somatic cell or somatic cell nucleus with an ooplastoid to create a nascent cell; (iii) activating the nascent cell; and (iv) culturing the nascent cell to obtain pluripotent non-embryonic stem cells.

In another embodiment of the present invention, the ooplastoid used in the method to generate pluripotent non-embryonic stem cells contains from about 10% to about 100% of the cytoplasmic volume of a mature oocyte.

In another embodiment of the present invention, the ooplastoid used in the method to generate pluripotent non-embryonic stem cells contains less than about 50% of the cytoplasmic volume of a mature oocyte.

In one embodiment of the present invention, the ooplastoid used in the method to generate pluripotent non-embryonic stem cells contains from about 17% to about 33% of the cytoplasmic volume of a mature oocyte.

In particular embodiments, the present invention is related to a method of producing pluripotent, non-embryonic stem cells wherein the somatic cell or somatic cell nucleus is a mature cell or where the somatic cell is an epithelial cell, lymphocyte or fibroblast.

In particular embodiments, the present invention is related to methods of producing pluripotent, non-embryonic stem cells where the somatic cell or somatic cell nucleus is combined with an ooplastoid to create a nascent cell by intracytoplasmic injection of the somatic cell nucleus into the zona free reduced volume ooplastoid; or where the somatic cell or somatic cell nucleus is combined with an ooplastoid to create a nascent cell by involves fusion induced by electrodes that are introduced directly into the culture dish and electrical pulses administered to the couplets immediately following micromanipulation; or where the somatic cell or somatic cell nucleus is combined with an ooplastoid to create a nascent cell by fusion in an electric field via electroporation; or fusion in a fusion chamber.

In particular embodiments, the present invention is related to methods of producing pluripotent non-embryonic stem cells comprising the following steps: (i) contacting one or more desired somatic cells or somatic cell nuclei with a super-ooplast derived from one or more enucleated zona pellucida free oocytes; (ii) dividing said super-ooplast into single nucleus containing nascent cells; (iii) activating the nascent cells; and (iv) culturing the nascent cells to obtain pluripotent non-embryonic stem cells.

In particular embodiments, the present invention is related to methods of producing pluripotent non-embryonic stem cells through using an enucleated zona pellucida free super-ooplast that comprises more than 100% of the cytoplasmic volume of a single egg and where the super-ooplast containing nuclei is partitioned into separate single nuclei containing nascent cells.

The present invention provides stem cells which are produced by the method of (i) contacting a desired somatic cell or somatic cell nucleus with an ooplastoid, wherein said ooplastoid is derived from an enucleated oocyte; (ii) combining said somatic cell or somatic cell nucleus with said ooplastoid to create a nascent cell, and (iii) culturing said nascent cell to obtain pluripotent non-embryonic stem cells.

The present invention provides a method of producing pluripotent non-embryonic stem cells comprising the following steps: (i) contacting a desired somatic cell or somatic cell nucleus with an ooplastoid, wherein the ooplastoid is derived from an enucleated oocyte; (ii) combining the somatic cell or somatic cell nucleus with the ooplastoid to create a nascent cell; and (iii) culturing the nascent cell to obtain pluripotent non-embryonic stem cells.

The present invention provides a method of producing pluripotent non-embryonic stem cells comprising the following steps: (i) contacting more than one desired somatic cells or somatic cell nuclei with an enucleated oocyte; (ii) dividing the oocyte somatic cell or oocyte somatic cell nuclei pairs into nascent cells, wherein each of the nascent cells contains a single nucleus; (iii) activating the nascent cells; and (iv) culturing the nascent cells to obtain pluripotent non-embryonic stem cells.

The present invention provides a method of producing pluripotent non-embryonic stem cells, wherein the cells are cultured on feeder layers comprising fibroblasts.

According to the present invention, the somatic cell or somatic cell nucleus used to produce nascent cells may be genetically modified prior to being used to generate pluripotent non-embryonic stem cells.

In particular embodiments, the present invention is related to methods of producing an ooplastoid comprising the following steps: (i) harvesting an oocyte from a female; (ii) maturing said oocyte to metaphase II; (iii) breaching or removing the zona pelucida of the metaphase II oocyte; (iv) enucleating the oocyte by removing the polar body and nuclear DNA of the oocyte through the breach of the zona pelucida or by oocyte partitioning; and (v) aspirating and pinching off an ooplastoid from the enucleated oocyte.

In particular embodiments, the zona pelucida is breached or removed using a chemical agent or using mechanical action.

In particular embodiments, the ooplastoid has from about 10% to about 100% of the volume from the original oocyte. In other embodiments, the ooplastoid has from about 15% to about 49% of the volume from the original oocyte. In a further embodiment, the ooplastoid has from about 17% to about 33% of the volume from the original oocyte.

### TERMS AND DEFINITIONS

The following terms are employed in the description of our invention:

Activation—refers to any materials and methods useful for stimulating a cell to divide.

Adult Stem Cells or “ASC's”—are certain cells found in vivo that are believed to be multipotent in nature. Use of the term “ASC's” refers to adult stem cells and multipotent stem cells.

Animals—non-human animal as used herein will be understood to include all vertebrate animals, except humans.

Autologous—refers to cells expressing the same major histocompatibility antigens (MHC) as the donor/source of the somatic cell used in the nuclear transfer process.

Cell—the term cell can refer to an oocyte, nascent cell, ES cell, an EC cell, a PNES cell, a P-PNES cell, a somatic cell or an early stage embryo.

Conditioned Growth Medium—refers to a growth medium that is further supplemented by factors derived from media obtained from cultures of feeder cells on which human PNES cells can be cultured.

Connective Tissue—connective tissue includes bone, cartilage, ligament, tendon, stroma and muscle.

Cryopreserved—the terms cryopreserving or cryopreserved as used herein refer to freezing a cell of the invention.

Enucleated—describes an object/cell from which the nucleus has been removed.

ES Cells—ES cells include embryonic stem cells and embryonic germ cells, and are believed to express the following characteristics: (i) the ability to divide in culture for an unlimited time and in an undifferentiated state, (ii) maintenance of a normal diploid karyotype, and (iii) pluripotency. Pluripotent ES cells are currently derived from embryos (naturally or via cloning) and/or fetal tissue as primary sources.

Euploidy—the state of karyotype comprised to a normal number of non-altered chromosomes (e.g., for humans, 46).

Growth Medium—growth medium means a suitable medium capable of supporting cell growth.

GV—gastro-vesicluar stage of Metaphase I maturation stage.

Immortality—Immortal cells are capable of continuous indefinite replication in vitro. As a practical matter, immortality is measured by observing continued proliferation of cells for longer than one year in culture.

Karyotype—a normal karyotype means that all chromosomes normally characteristic of the species are present and have not been noticeably altered.

Maturation Period—the time period beginning with aspiration of the immature oocyte from either human or animal ovarian follicles and including the time spent maturing the oocytes in a maturation medium prior and lasting until the oocyte attains a certain maturation endpoint, such as metaphase II, but not limited to metaphase II.. The maturation endpoints relevant to the present invention include germinal vesicle stage (P1) or (GV) metaphase I (M1), metaphase II (MII), and post-activation oocytes.

Multipotent Stem Cells—these are stem cells that are found in mature animals/humans and which are believed to be capable of differentiating into cells derived from some, but not all, embryonic germ layers. Use of the term “ASC's” refers to adult stem cells and multipotent stem cells.

Metaphase I Immature Oocytes—refers to the stage of development known as Metaphase 1 of meiosis.

Nascent Cell—the nascent cell is produced as a result of the fusion or injection of an individual somatic cell or cell nucleus with an ooplastoid. The P-PNES described herein are considered examples of nascent cells.

Oocyte—the egg cell, a specialized cell that carries one half the normal number of chromosomes (haploid) and is surrounded a thick layer of glycoproteins and extracellular matrix material called the zona pellucida. In humans, the oocyte carries 23 chromosomes.

Oocytoid—Oocytoids arise after multiple nuclei are inserted or fused into an ooplast or super-ooplast, and by fragmenting such multinucleated ooplasts or super-ooplasts into single nucleus containing nascent cells (oocytoids).

Ooplasts—Ooplasts result from the enucleation of an oocyte. Ooplasts are enucleated, plasma-membrane enclosed, zona pellucida intact or zona pellucida free oocytes.

Super-ooplasts—result from the fusion of two or more ooplasts or (enucleated oocytes). Super-ooplasts of greater than 100% of the volume of a single oocyte may also be created by fusing an enucleated oocyte with blasts containing fluids other than ooplasm.

Ooplastoids—Ooplastoids result from the partitioning of an oocyte or ooplast. Ooplastoids are enucleated, plasma-membrane enclosed, zona pellucida free portions of the oocyte.

Ooplastoid/Somatic Cell Couplet—the ooplastoid/somatic cell couplet refers to the aggregated individual somatic cell with an individual ooplast in a 1:1 ratio and prior to fusion to form the Nascent Cell.

Prophase 1 Immature Oocytes—refers to the stage of development known as prophase 1 stage of meiosis or typically referred to as GV or germinal vesicle stage oocytes.

Pluripotent—refers to cells that have the potential to develop into cells derived from all three embryonic germ layers (mesoderm, endoderm and ectoderm) of animals/humans but which do not have the ability to form into a complete human being/animal.

PNES or PNES Cells—pluripotent non-embryonic/non-fetal tissue derived stem cells that are pluripotent and can proliferate in culture indefinitely and in an undifferentiated state.

P-PNES or P-PNES Cells—precursors to PNES that are nascent cells.

Progenitor or Precursor Cells—immature cells that can differentiate into a limited number of different cells of the same tissue type, for example a lymphoid progenitor cell can differentiate into any one of he following: T-cells, B-cells or natural killer cells.

SCID Mouse—a mouse or mouse strain with severe combined immunodeficiency (SCID) that displays profound defects in both humoral and cellular immunity.

Somatic Cells—cells of the body carrying a diploid set of chromosomes. In humans, somatic cells carry 46 chromosomes.

Specific Differentiated Cells—are cells derived as a result of directing PNES or ES to become multipotent/adult stem cells, and then further directing those multipotent/adult stem cells to differentiate into Specific Differentiated Cells found in animals and humans that do not have the ability to further differentiate. Examples include sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc.

Stem Cells—all forms of stem cells have two characteristics that separate them from other cells. First, they are able to divide and replace themselves for indefinite periods. Second, at the same time that stem cells are replacing themselves they can produce cells capable of differentiating into other more specialized cells

Stem Cell Markers—stem cell markers are cell surface molecules, usually glycoproteins, which are characteristic of a particular type of stem cell. Different stem cell lineages express unique arrays or patterns of markers that are detected using monoclonal antibodies which specifically recognize and bind to the markers.

Totipotent Cells—cells that have the ability to develop into cells derived from all three embryonic germ layers (mesoderm, endoderm and ectoderm) and an entire organism (e.g., human being if placed in a woman's uterus in the case of humans). Totipotent cells may give rise to an embryo, the extra embryonic membranes and all post-embryonic tissues and organs.

Undifferentiated—an undifferentiated cell is also an unspecialized cell that retains the potential for differentiating into other more specialized cells

Zona Pellucida Free—refers to an oocyte, oocytoid, ooplast, or an ooplastoid from which the zona pellucida has been removed.

As used herein and in the appended claims, the singular forms “a,” “an,” and “the,” include plural referents unless the context clearly indicates otherwise. Thus, for example, reference to “a cell” includes one or more of such cells or a cell line derived from such a cell, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

### BRIEF DESCRIPTION OF THE DRAWINGS

FIG. 1 demonstrates micromanipulation of the mature mouse metaphase II oocyte: A) the mouse oocyte is oriented on the micromanipulators, B) the polar body and underlying cytoplasm containing the nuclear DNA is removed, C) formation of the enucleated oocyte is acheived by partitioning, D) shows how one mouse oocyte may be partitioned into three enucleated ooplastoids (bottom right arrow), the zona pellucida (center arrow) which is discarded, and the polar body and nuclear DNA (top center arrow) which are discarded. Bar=100 μm

FIG. 2 shows micromanipulation and electrofusion of the ooplastiod/somatic cell couplet: A) demonstrates introduction of the somatic cell to the enucleated ooplastoid, B) shows establishing firm membrane-to-membrane contact between the ooplastoid/somatic cell couplet by pressing the somatic cell against the ooplastoid, C) shows one ooplastoid/somatic cell couplet prior to electrofusion, D) shows one ooplastoid/somatic cell couplet positioned between the electrodes in an electroporation chamber. Bar=100 μm

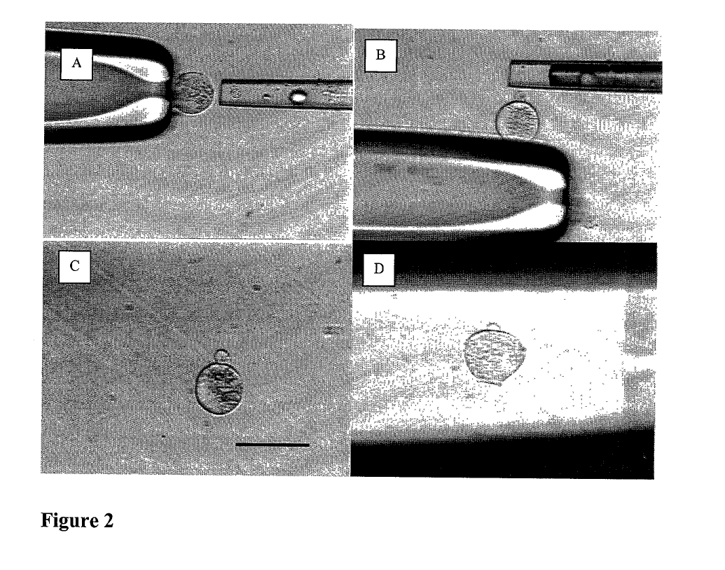

FIG. 3 shows the results of mitotic cell division of nascent cells at 72 h post nuclear transfer for both bovine and murine systems: A) a bovine nascent cell formed by electrofusion of an ooplastoid and a somatic cell has mitotically divided to form approximately 12 P-PNES cells, and B) a mouse nascent cell formed by the injection of a somatic cell into an ooplastoid has mitotically divided to form 8 P-PNES. Bar=100 μm.

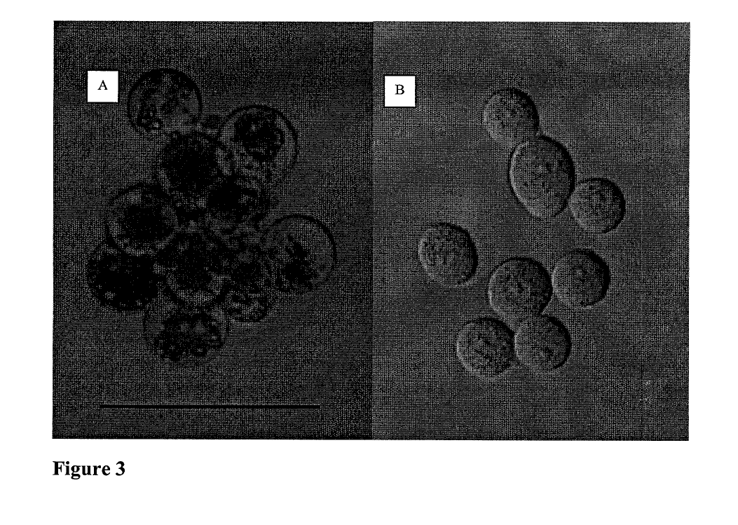

### DETAILED DESCRIPTION OF THE INVENTION

Pluripotent Non-Embryonic, Non-Fetal Tissue Stem Cells (PNES)

The present invention provides a new source for obtaining pluripotent stem cells and stem cell lines. This invention does not require the use, creation and/or destruction of embryos or fetal tissue and does not in any way involve creating a cloned human or animal or the mixing of materials or cells between/among species. The products of this invention are pluripotent non-embryonic, non-fetal derived stem cells (PNES) and stem cell lines.

To create PNES cells, portions of the oocyte cytoplasm (“ooplastoids”) are produced and combined with nuclear material of individual somatic cells. Subsequently, the newly formed P-PNES/nascent cells are cultured and give rise to PNES cells and PNES cell colonies. The oocytes and/or ooplastoids utilized in this procedure never become fertilized and never develop into embryos.

More specifically, this invention provides (a) methods of creating and culturing P-PNES cells to yield purified PNES cells which have the ability to differentiate into cells derived from mesoderm, endoderm, and ectoderm germ layers, (b) methods for maintaining and proliferating PNES cells in culture in an undifferentiated state for greater than one year, and (c) the use of those PNES cells for scientific and therapeutic purposes. These applications include, but are not limited to, use of PNES cells in (a) scientific discovery and research involving cellular development and genetic research, (b) drug development and discovery (e.g., screening for efficacy and toxicity of certain drug candidates and chemicals), (c) gene therapy (e.g., as a delivery device for gene therapy), and (d) tissue/cellular regeneration and replacement therapies and applications (e.g., replacement of damaged or destroyed blood cells, cardiac muscle, neural cells destroyed by Parkinson's, liver cells, etc.). Set forth in the remainder of this section is a detailed description of the steps and inventions described in the prior sentences.

It is important to note that this invention provides a method for deriving P-PNES cells and PNES cell lines involving unique techniques and methods, including the nuclear transfer of genetic material from a somatic cell into an enucleated, plasma membrane enclosed, zona pellucida free human ooplastoid having from 10% to 100% of the volume of ooplasm of the original egg. For description of previously reported nuclear transfer techniques, refer Campbell et al, Theriogenology, 43:181 (1995); Collas et al, Mol. Report Dev., 38:264-267 (1994); Keefer et al, Biol. Reprod., 50:935-939 (1994); Sims et al, Proc. Natl. Acad. Sci., USA, 90:6143-6147 (1993); WO 94/26884; WO 94/24274, and WO 90/03432, which are incorporated by reference in their entirety herein. Also, U.S. Pat. Nos. 4,944,384; 4,664,097; and 5,057,420 describe procedures for nuclear transplantation.). The present invention for nuclear transfer differs from those previously published in the literature in several significant ways. First, the inventor hereof was the first to announce the use of a technique wherein the zona pellucida of the oocyte used in the invention is avoided in the process of somatic cell nuclear transfer to create PNES cells. (M. J. Levanduski, Nuclear Transfer Procedure for the Production of Human Stem Cell Cultures Without Creating Embryos, 2001 International Workshop on Human and Therapeutic Cloning, Mar. 9, 2001 (In Press)). Subsequent to the cited report of the inventor, two other reports have been published which indicate that others are working with similar zona pellucida free techniques. A critical distinction is that the present invention involves a zona pellucida free somatic cell nuclear transfer technique that does not attempt to create an embryo. The reports cited below involve a zona pellucida free nuclear transfer technique in which the objective is to create a cloned embryo. See Simplification of Bovine Somatic Cell Nuclear Transfer by Application of Zona-Free Manipulation Technique (2001), P. J. Booth, S. J. Tan, R. Reipurth, P. Holm, H. Callesen, Cloning and Stem Cells, Vol. 3:3, 139-150; Somatic Cell Cloning Without Micromanipulators, G. Vajta, I. M. Lewis, P. Hyttel, G. A. Thouas, and A. O. Trounson (2001), Cloning, Vol. 3:2, 89-95.) Second, the present invention provides that after enucleation, the oocyte is subdivided into up to 6 membrane intact ooplastoids, having anywhere from about 10% to about 100% of the total volume of the original oocyte. Previous nuclear transfer procedures directed to creating viable cloned embryos generally utilized enucleated recipient ooplasts consisting of from about 50% to about 100% of the oocytes original volume in order to maximize ooplasm/somatic cell v/v ratio. Third, the conditions of intracytoplasmic nucleus injection, electroporation, and cell fusion (somatic cell to ooplastoid) in the present invention varies significantly compared to standard fusion techniques. In the current invention, the basic unit, ooplastoid/somatic cell aggregate, is not enclosed by a zona pellucida and therefore is very fragile and is subject to damage very easily. Fusion of the ooplastoid/somatic cell aggregate using a standard fusion chamber is described in the present invention. Accordingly, the present invention also discloses a unique fusion technique involving moveable electrodes that are introduced directly into the micromanipulation Petri dish where the ooplastoid/somatic cell aggregate is assembled and immediately electroporated to induce fusion. The present invention provides for optimized fusion and activation parameters and the resulting nascent cells (P-PNES) for all species. Finally, the techniques utilized for directing mitotically dividing P-PNES cells to become PNES cells in in vitro culture is herein unique

Finally, the techniques utilized for directing mitotically dividing P-PNES cells to become PNES cells in in vitro culture is herein unique. The inventor first reported this technique in 2001 (Procedure for the Production of Human Stem Cell Cultures Without Creating Embryos, M.Levanduski, 2001 International Workshop on Human and Therapeutic Cloning, Mar. 9, 2001. in press). A similar technique was recently reported, however this technique in bovine involved culture of pooled embryo blastomeres to create bovine ES cells (Pluripotency of Bovine Embryonic Cell Line Derived from Precompacting Embryos. M. Mitalipova, Z. Beyhan, and N. L. First, 2001,Cloning, vol 3, no. 2, pages 59-68.)

Source, Maturation and Preparation of Oocytes

There are several actual or potential sources for human oocytes for this invention and the application thereof. First, immature human oocytes are obtained from established human in vitro fertilization centers with appropriate patient knowledge and consent. (The oocytes obtained via this channel are immature eggs that would otherwise be discarded. Generally human IVF patients produce approximately 10-12 oocytes per cycle, approximately 80% of which are mature metaphase oocytes capable of becoming fertilized and forming an embryo for the patient. The remaining oocytes (approximately 20%) are immature (prophase I or metaphase I) oocytes. Immature human oocytes are not capable of fertilization or creating an IVF embryo at that point and are therefore typically discarded as medical waste by the IVF laboratory).

A second source for human oocytes may be via a dedicated oocyte donor who donates her oocytes for a specific application for a friend or relative (e.g., a sister of a patient with a degenerative disease). A third source would be obtaining of oocytes via purchase from willing donors in conformity with all applicable laws and regulations.

Immature (prophase I and metaphase I) donated oocytes undergo a maturation period in specialized medium until the oocytes attain the metaphase II stage. This period of time beginning with aspiration of the immature oocyte from the ovarian follicles and including the time spent maturing the oocytes in a maturation medium and lasting until the oocyte attains the metaphase II stage is known as the maturation period. Only human oocytes which mature in vitro to the metaphase II stage within 36 h of oocyte retrieval are utilized further in the current invention.

The maturation period of the oocytes will depend on the initial stage of development of the oocyte and end stage of development desired for use. Accordingly, the oocytes are incubated for a fixed time maturation period, which ranges from about 10 to 48 h. Alternatively, the oocytes can be matured for any period of time: an oocyte can be matured for greater than 10 h, matured for greater than about 20 h, matured for greater than about 24 h, matured for greater than about 36 h, more preferably matured for greater than 48 h, even more preferably matured for greater than about 53 h, preferably matured for greater than about 60 h, preferably matured for greater than about 72 h, or matured for greater than about 90 h. The term “about” with respect to oocyte maturation can refer to plus or minus 3 h.

The present invention provides non-embryonic stem cells and methods of making them from a starting material comprising human or non-human animal oocytes. In a preferred embodiment of the present invention the source of oocyte is a human female. In certain embodiments of the present invention, the non-human animal species providing oocytes is bovine. In other embodiments, the non-human animal species providing oocytes is ovine. In still other embodiments, the non-human animal species providing oocytes is porcine. In yet other embodiments, the non-human animal species providing oocytes is caprine. Other non-human animals contemplated for providing oocytes for use in the present invention include, but are not limited to, horses (equine), dogs (canine), cats (feline), buffaloes, llamas, ferret, guinea pig, rabbits and other commercial and domestic species.

Animal oocytes were and will be secured from reputable commercial suppliers. Maturation of the oocytes followed a known standard procedure. For example, immature oocytes may be washed in HEPES buffered embryo culture medium (HECM) as described in Seshagine et al., Biol. Reprod., 40, 544-606, 1989, and then placed into drops of maturation medium consisting of tissue culture medium (TCM) 199 containing 10% fetal calf serum which contains appropriate gonadotropins such as luteinizing hormone (LH) and follicle stimulating hormone (FSH), and estradiol under a layer of lightweight paraffin or silicon at 39 C.

An alternative source for murine oocytes is via collection from mice stimulated by exogenous hormones. Mouse oocytes were obtained by inducing superovulation of 4-8 week old females (B6CBA/F1, Jackson Lab) by first administering intraperitoneal (IP) injections of 5 IU Pregnant Mare Serum Gonadotropin, (Calbiochem 367222) followed by 5 IU of hCG (Sigma). Next, the mice were sacrificed at 22 h post hCG injection and the ovaries and fallopian tubes were dissected to remove oocytes. The recovered oocytes were then washed in HECM (Conception Technologies, EH500) supplemented with 10% Plasmanate (Bayer, Elkhart, Ind.). Granulosa cells were removed from the oocyte preparation by treatment of 0.5-1.0 mg/ml hyaluronidase (Sigma 40K8927) followed by mechanical pipetting of the cells using a fine bore Pasteur pipette. The denuded oocytes were washed in HECM prior to micromanipulation to remove hyaluronidase residue. Only mature Metaphase II oocytes were utilized further in this procedure.

After maturation, but prior to enucleation, the oocytes of all species described here are denuded of surrounding granulosa cells by using a chemical treatment of HECM containing 0.5 to 1.0 mg/ml of hyaluronidase (Sigma H3757). Subsequent repeated pipetting through very fine bore pipettes or by vortexing briefly mechanically removes the granulosa cells. The denuded oocytes are then screened for maturation status and the selected metaphase II oocytes, as determined by the presence of polar bodies, are then used for nuclear transfer. Next, the oocytes are enucleated.

Enucleation of Mature Metaphase II Oocytes

The nucleus of the oocyte (human and animal) can be removed by standard techniques, such as described in U.S. Pat. No. 4,994,384, which is incorporated by reference herein. For example, metaphase II oocytes are placed in HECM, optionally containing 7.5-15.0 μg/ml Cytochalasin B (Sigma C6762), for immediate enucleation using micromanipulation procedures.

Enucleation may be accomplished microsurgically using a micropipette to remove the polar body and the adjacent cytoplasm after breaching the zona pellucida. The oocytes may then be screened to identify those oocytes that have been successfully enucleated. This screening may be effected by staining the oocytes with 1-5 mg/ml Hoechst 33342 dye in HECM, and then viewing the oocytes with a microscope equipped with ultraviolet irradiation for less than 10 seconds. The oocytes that have been successfully enucleated are then placed in a suitable culture medium e.g., CR2 medium (CR1 medium supplemented with amino acids), the latter of which is described in U.S. Pat. No. 5,096,822, “Bovine embryo medium,” Rosenkrans Jr. et al., Nov. 3, 1992, hereby incorporated herein by reference in its entirety, including all figures, tables, and drawings. One of skill in the art would understand that a variety of culture media are used depending on the species and cell type being cultured.

The zona pellucida of the mammalian oocyte may be breached and/or removed by mechanical breaching and/or chemical breaching. Mechanical breaching and/or removal of the zona pellucida is accomplished by cutting the zona with a fine glass or metalic needle or equivalent. Chemical breaching and/or removal of the zona pellucida is accomplished by treatment with Acidic Tyrodes solution, or by treatment with a wide variety of proteases such as Pronase. Localized application of the chemical may result in a zona breach (hole) whereas treatment of the entire oocyte may result in complete dissolving of the zona pellucida.

In another method of enucleation, a glass needle (micropipette) is placed into an oocyte and the nucleus is aspirated into the needle. Thereafter, the needle can be removed from the oocyte without rupturing the plasma membrane. See, U.S. Pat. No. 4,994,384; U.S. Pat. No. 5,057,420; and Willadsen, 1986, Nature 320:63-65. An enucleated oocyte is preferably prepared from a mature metaphase II oocyte that has been matured for greater than 24 h, preferably matured for greater than 36 h

In the present invention, the recipient oocytes are enucleated at a time ranging from about 10 h to about 48 h after the initiation of maturation, more preferably from about 10 h to about 36 h after initiation of maturation, more preferably from about 16 h to about 24 h after initiation of maturation, and most preferably about 16 to about 18 h after initiation of maturation.

Ooplastoid Generation

The process of ooplastoid generation in the present invention is a novel technique for the following reasons. First, in a certain embodiment of the present invention enucleated oocytes are subdivided to create plasma membrane-contained ooplastoids that have a significantly smaller volume than an intact oocyte, thus allowing the creating of multiple ooplastoids from a single oocte. In a preferred embodiment the ooplastoid has a volume of less than 50% of a whole oocyte. More particularly, the ooplastoids have a volume from about 17% to about 33% of a whole oocyte. Second, the ooplastoid is not enclosed by a zona pellucida. There are several methods of creating these reduced volume ooplastoids. Some examples include, but are not limited to, the following:

1. Enucleated oocytes are placed in HECM containing 7.5-15.0 μg/ml Cytochalasin B. Next, the enucleated oocytes are microsurgically subdivided using micropipettes and a micromanipulation apparatus (Narashige, Japan). A portion of each enucleated oocyte is aspirated and pinched off from the oocyte leaving the ooplast plasma membrane intact. The procedure is repeated until the enucleated oocyte is subdivided into 2-6 ooplastoids, with each enucleated ooplastoid containing from about 17% to about 50% of the original volume of the intact oocyte. The ooplastoid generation procedure is repeated for each enucleated oocyte. Through this process the zona pellucida is left behind as a waste product and plays no further role in the invention.

2. In some circumstances, it may be advantageous for the ooplastoids to retain as much of the volume of the original oocyte as possible, therefore only one oocyte would yield one ooplastoid and the volume would be from about 50% to about 100% of the volume of the original oocyte.

3. The zona pellucida of the nucleated or enucleated whole oocyte may be removed chemically using standard techniques such as protease, or acidic Tyrodes solution. The zona pellucida free oocytes are placed in HECM containing 7.5-15.0 μg/ml Cytochalasin B. The zona pellucida free oocyte is then subdivided using micropipettes and a micromanipulation apparatus (Narashige, Japan). A portion of each oocyte is aspirated and pinched off from the oocyte leaving the plasma membrane intact. In one embodiment of the invention, the procedure is repeated until the enucleated oocyte is subdivided into 2-6 plasma membrane contained ooplastoids. Ooplastoids are then screened by staining with 1-5 μg/ml Hoechst 33342 dye in HECM, and then viewing the ooplastoids with a microscope equipped with ultraviolet irradiation for less than 10 seconds. Only enucleated ooplastoids are utilized further.

In one embodiment, each ooplastoid contains less than 100% of the original volume of the oocyte; preferably each ooplastoid contains less than about 50% of the original volume of the oocyte. Alternatively, each ooplastoid contains less than about 30% of the original volume of the oocyte. Alternatively, each ooplastoid contains less than about 20% of the original volume of the oocyte. In another embodiment, each ooplastoid contains from about 10% to about 100% of the original volume of the oocyte. Preferably, each ooplastoid contains from about 15% to about 50% of the original volume of the oocyte. More preferably, each ooplastoid contains from about 15% to about 37% of the original volume of the oocyte. Even more preferably, each ooplastoid contains from about 17% to about 33% of the original volume of the oocyte. The ooplastoids can be human or animal ooplastoids.

Source of Somatic Nucleus

The ooplastoids generated above will be combined through the process of nuclear transfer with chosen somatic cells. The somatic cells in the current invention are human as well as other animal species, however it is important to reiterate that the current invention involves combining somatic cells' or somatic cells nuclei with ooplastoids of the same species, i.e. human-to-human, mouse-to-mouse, bovine-to-bovine. The human or animal somatic cells may be obtained by well-known methods. The cells used for nuclear transfer may be obtained from different organs, e.g., skin, lung, pancreas, liver, stomach, intestine, heart, reproductive organs, bladder, kidney, urethra and other urinary organs, etc., generally from any organ or tissue containing live nucleated somatic or diploid germ cells. Human and animal cells useful in the present invention include, by way of example, adult stem cells, sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. generally any live nucleated somatic or diploid germ cell. These are just examples of suitable donor cells The somatic cells utilized in the present invention are granulosa cells of bovine, ovine, murine, or human origin.

Preparation of the Donor/Host Somatic Cell

The human or animal somatic cells utilized in the current invention are cultured in vitro prior to nuclear transfer. In the present invention prior to nuclear transfer the human and animal somatic (granulosa) cells are cultured in ECM supplemented with standard (10%) or alternatively reduced 0.5% concentrations of FCS or Plasmanate (Bayer). It may be necessary to induce quiescence in donor cells prior to nuclear transfer, using a suitable technique known in the art. The techniques for stopping the cell cycle at various stages have been summarized in U.S. Pat. No. 5,262,409, which is herein incorporated by reference in its entirety. For example, while cycloheximide has been reported to have an inhibitory effect on mitosis (Bowen and Wilson (1955) J. Heredity 45: 3-9), it may also be employed for improved activation of mature bovine follicular oocytes when combined with electric pulse treatment (Yang et al. (1992) Biol. Reprod. 42 (Suppl. 1): 117).

Combining Somatic Cell/Nucleus with the Ooplast/Ooplastoid

In a preferred embodiment of the present invention, one individual somatic cell nucleus is transferred into one ooplastoid (a 1:1 ratio) to produce a P-PNES cell which is a nascent cell. It is important to state that the current invention involves transferring a somatic cell into an ooplastoid of the same species (i.e. human somatic cell fused to human ooplastoid, murine somatic cell to murine ooplastoid, bovine somatic cell to bovine ooplastoid, etc.). Nuclear transfer techniques are utilized in the current invention include (a) direct intracytoplasmic injection of the somatic cell nucleus into the enucleated ooplastoid, and (b) electrofusion of the entire somatic cell to the enucleated ooplastoid Both of these techniques are utilized in human and animal species for the current invention.

Direct intracytoplasmic injection of the somatic cell nucleus into the enucleated ooplast is well known in the art of nuclear transfer. This technique is disclosed in Collas and Barnes, Mol. Reprod. Dev., 38:264-267 (1994), and incorporated by reference in its entirety herein. Briefly this involves breaking the outer membrane of the somatic and injecting the nucleus directly into the enucleated ooplast. This is accomplished utilizing an injection micropipette with a diameter smaller than the diameter of the somatic cell, thereby rupturing the somatic cell plasma membrane prior to injection of the nucleus into the enucleated ooplast. The result is that the somatic cell nucleus is effectively transferred into the intact enucleated ooplast. Activation of the oocyte may occur as a result of the intracytoplasmic injection treatment, or may be intentionally effected shortly thereafter, typically less than 24 h after injection.

The present invention provides a method where individual somatic cells and ooplastoids are fused by electrofusion. Electrofusion is accomplished by providing a pulse of electricity that is sufficient to cause a transient breakdown of the plasma membrane. This breakdown of the plasma membrane is rapid and the membrane subsequently reforms. Basically, if two adjacent membranes are induced to breakdown and upon subsequent reformation the lipid bilayers will intermingle and small channels will open between the two independent cells. As a consequence, and due to the thermodynamic instability of such a small opening, the channels will enlarge until the two cells become one. See U.S. Pat. No. 4,997,384 to Prather et al., for a further discussion of this process, which is hereby incorporated by reference in its entirety. A variety of electrofusion media can be used including e.g., sucrose, mannitol, sorbitol and phosphate buffered solution.

Electrofusion in the present invention is described in which somatic cells are successfully fused to ooplasts/ooplastoids using a commercially available fusion chamber and defined electrofusion parameters and media. It should be noted however, that using a commercially available fusion chamber can result in reduced fusion efficiency due to handling of the fragile zona pellucida free ooplastoid, somatic cell, or the ooplastoid/somatic cell couplet. Despite reduced survival and fusion efficiency of this process, successful fusion and post fusion cleavage have been achieved and described herein. For example, the human or animal cell and same species ooplastoid may be fused in a 500 μm chamber by application of an electrical pulses of 90-120 V for about 25 μsec/pulse. After fusion, the resultant fused P-PNES/nascent cells are then placed in a suitable medium. Activation of the ooplastoid may occur as a result of the electroporation treatment, or may be intentionally effected shortly thereafter, typically less than 24 h after fusion.

The present invention also includes an alternative electrofusion technique comprising micromanipulation of the cells and electroporation without a commercially produced electrofusion chamber. Instead the ooplastoids and somatic cells are placed in a Petri dish, or equivalent culture dish, containing fusion medium. Micropipettes are introduced and each somatic cell is paired with a single ooplastoid to create an ooplastoid/somatic cell couplet. Electrodes are then immediately introduced directly into the Petrib dish, and electrical pulses are administered immediately to the couplets. The distance between the electrodes, the voltage of the pulse, the duration of the pulse, and the number of pulses are factors that are influence survival of the cells and fusion success. Those of skill in the art will appreciate that optimization of fusion parameters using this system will depend on the particular species being fused, the type and size of ooplastoid, and the type of donor cell.

Activation of Ooplastoids, P-PNES Cells and PNES Cells

After combination of the somatic cell nucleus with the enucleated ooplastoid by injection or electrofusion, activation of the resulting P-PNES/nascent cells may be required to stimulate development. Activation is required for human, bovine, ovine, and murine ooplasts and/or P-PNES/nascent cell, however the timing and/or technique may differ between species. One method of activation known in the art involves electrical pulses and this method is sometimes sufficient for activation of cells. The ooplastoid and or P-PNES/nascent cell may have become “activated” as a result of the intracytoplasmic injection procedure or as a result of the electrofusion procedure, in which case no additional activation treatment is required. If additional activation treatment is required, electroporation treatments may be applied. For example, the human or animal P-PNES/nascent cell may be pulsed in a 500 μm chamber by application of repeated electrical pulses of 90-120 V for about 25 μsec/pulse.

Alternatively, other non-electrical means for activation are useful and are often necessary for proper activation of an ooplastoid or P-PNES/nascent cell. See, e.g., Grocholova et al., 1997, J. Exp. Zoology 277: 49-56; Schoenbeck et al., 1993, Theriogenology 40: 257-266; Prather et al., 1989, Biology of Reproduction 41: 414-418; Prather et al., 1991, Molecular Reproduction and Development 28: 405-409; Mattioli et al., 1991, Molecular Reproduction and Development 30: 109-125; Terlouw et al., 1992, Theriogenology 37: 309; Prochazka et al., 1992, J. Reprod. Fert. 96: 725-734; Funahashi et al., 1993, Molecular Reproduction and Development 36: 361-367; Prather et al., Bio. Rep. Vol. 50 Sup 1: 282; Nussbaum et al., 1995, Molecular Reproduction and Development 41: 70-75; Funahashi et al., 1995, Zygote 3: 273-281; Wang et al., 1997, Biology of Reproduction 56: 1376-1382; Piedrahita et al., 1989, Biology of Reproduction 58: 1321-1329; Machaty et al., 1997, Biology of Reproduction 57: 85-91; and Machaty et al., 1995, Biology of Reproduction 52: 753-758.

Examples of components that are useful for non-electrical activation include ethanol; inositol trisphosphate (IP₃); divalent ions (e.g., addition of Ca²⁺ and/or Sr²⁺); ionophores for divalent ions (e.g., the Ca²⁺ ionophore ionomycin); protein kinase inhibitors (e.g., 6-dimethylaminopurine (DMAP)); protein synthesis inhibitors (e.g., cyclohexamide); phorbol esters such as phorbol 12-myristate 13-acetate (PMA); and thapsigargin. It is also known that temperature change and mechanical techniques are also useful for non-electrical activation. The invention includes any activation techniques known in the art. See, e.g., U.S. Pat. No. 5,496,720, entitled “Parthenogeneic Oocyte Activation,” issued on Mar. 5, 1996, Susko-Parrish et al., and Wakayama et al., 1998, Nature 394: 369-374, each of which is incorporated herein by reference in its entirety, including all figures, tables and drawings.

When ionomycin and DMAP are utilized for non-electrical activation, ionomycin and DMAP may be introduced to cells simultaneously or in a step-wise addition, the latter being a preferred mode as described herein. Preferred concentrations of ionomycin and DMAP are 0.5 μM ionomycin to 50 μM ionomycin and 0.5 mM DMAP to 50 mM DMAP, more preferably 1 μM ionomycin to 20 μM ionomycin and 1 mM DMAP to 5 mM DMAP, and most preferably about 10 μMolar ionomycin and about 2 mM DMAP, where the term “about” can refer to plus or minus 2 μM ionomycin and 1 mM DMAP.

Culture Conditions of PNES or P-PNES cells, and Prevention of Cell Clumping

P-PNES/nascent cells of all species produced by somatic cell nuclear transfer described here are cultured in ECM (Quinns Advantage Cleavage Medium, Sage Biopharma, Bedminster, N.J.) supplemented with 10% Plasmanate(Bayer), HSA, or FCS at 5-6% CO2 at 37° C. Each P-PNES/nascent cell in this invention is cultured individually for 72-96 h. P-PNES cells are observed using an inverted Nikon Eclipse microscope with a heated (37° C.) stage at 24, 48, 72, and 96 h post micromanipulation/activation. In the human, mouse, and bovine each P-PNES/nascent cell cleaves (divides mitotically) to form two to four separate cells at about 24 h post activation, four to eight separate cells at about 48 h post activation, and eight or more cells at about 72 and about 96 h. Dividing cells at 72 to 96 h post activation may begin to form plasma membrane contact between adjacent cells. To prevent formation of cell to cell membrane connections, the cells are separated by mechanical (pipetting) treatment and chemical treatment with hyaluronidase, trypsin, chymotrypsin or similar chemical treatment in calcium and magnesium free phosphate buffered saline with 10% FCS. Mechanically separated cells originating from different P-PNES/nascent cells may be pooled at about 72 to 96 h post activation. If the pooled P-PNES/nascent cells all originated from the same somatic cell donor/source then the pooled cells are presumably autologous to each other as well as the somatic cell donor/source.

Culture Conditions of P-PNES Cells for Formation of PNES Cells

For human, mouse, and bovine cells, 100 to 200 pooled P-PNES cells about 72-96 hour post activation are introduced to a fibroblast feeder culture system as follows. Mouse or other animal fetal fibroblasts are isolated from postcoitum fetuses. Human fibroblasts may originate from a patient or from a screened donor. Mitomycin or ultra-violet inactivated fibroblasts are cultured in monolayers at 70,000 to 90,000 cells/cm² in Nunc 35×10 mm culture dishes, in DMEM supplemented with 10% FCS, L.I.F., and S.I.T. (Sigma), with 5-6% CO2 at 37° C. Disaggregated, pooled P-PNES cells about 72-96 hour post activation are introduced and spread upon the inactivated fibroblast monolayer using a sterile Pasteur pipette. Cells are observed periodically for the next 48 h and mechanically disaggregated using a Pasteur pipette if clumps of cells are observed. This is repeated until cells are observed to adhere to the feeder layer. On about day 3-7 after introducing the cells to the feeder layer the cell colonies are observed for mechanical cell sorting. Cells on the monolayer are manipulated using an inverted microscope equipped with a micromanipulator and a polished 25 μm micropipette. Alternatively, a hand drawn sterile Pasteur pipette may be used to mechanically manipulate cultured cells while the technician is viewing the cell colonies with a stereomicroscope. Cells exhibiting embryonic stem cell like morphology (i.e., flat round or irregular shape, form loose aggregates, can form embryoid bodies) are selected and physically separated from the monolayer and aspirated into the micropipette or Pasteur pipette. See U.S. Pat. No. 6,200,806 and Thompson, J. A. et al. Science, 282:1145-7, 1998. See also Amit, M., Thompson, J. A. et. al. Clonally Derived Human Embryonic Stem Cell Lines Maintained Pluripotency and Proliferative Potential For Prolonged Periods of Culture. Dev. Biol. 227, 271-278 (2000). The selected cells are then transferred (passaged) to a new inactivated fibroblast feeder layer for continued culture. As mentioned above, these cells are referred to as pluripotent non-embryonic/non-fetal tissue derived stem cells or PNES cells. PNES cells are passaged to fresh inactivated mouse fetal fibroblast monolayer cultures about every 7-10 days according to standard embryonic stem cell culture techniques. Aliquots of these PNES cells may be characterized as stem cells using the stem cell markers. For human PNES cells are SSEA-1(−).SSEA-3(+).SSEA-4(+).TRA1-60(+).TRA-1-81(+). The cells are to be tested using immunofluorescent microscopy. The mouse monoclonal antibodies to stage-specific embryonic antigens (SSEA) 1.3 and 4 are available from Hybridoma Bank at NIH. TRA-1-60 and TRA-1-80 are available from Vector Laboratories. To certify PNES cells for the presence or absence of the indicated markers, the cells will be placed on the cover slips pre-treated with poly-lysine or containing irradiated mouse embryonic fibrolasts (3000 rad) allowed to adhere and spread and fixed with 4% formalin. Following the fixation the cells are be stained with different antibodies and the presence of the marker is identified by binding the FITC labeled rabbi anti-mouse polyclonal antibodies. As a control the embryocarcinoma (EC) cell line NTERA-2 cl. D1 (available from ATCC) will be used.

Culture of human derived, pooled 72-96 h post activation P-PNES cells may be performed in a manner identical to that described for the mouse and bovine pluripotent ES cells. This involves using mouse fetal fibroblast monolayers as described above, a disadvantage if the cells are ultimately destined for use in cell replacement clinical therapy. Alternatively, human fibroblast monolayers may be substituted. The source of the human fibroblasts used for the continuous PNES cell culture ideally will be autologous to the source of the somatic cell used for nuclear transfer.

When grown in culture, pluripotent ES cells, and therefore PNES cells, may be inhibited from differentiation by growth on inactivated fibroblast feeder layers. Methods for isolating one or more cells from another group of cells are well known in the art. See, e.g., Culture of Animal Cells: a manual of basic techniques (3rd edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; and Animal Cells: culture and media, 1994, D. C. Darling, S. J. Morgan John Wiley and Sons, Ltd.

PNES cells may be maintained in cell culture using an appropriate growth medium. PNES cell growth or culture medium means any medium that supports growth of PNES cells in culture. For example, the present invention may be practiced using a variety of human PNES cell growth media prepared on a base of Dulbecco's minimal essential media (DMEM) supplemented with 15% fetal calf serum, 2 mM glutamine, 1 mM sodium pyruvate, or glucose and phosphate free modified human tubal fluid media (HTF) supplemented with 15% fetal calf serum, 0.2 mM glutamine, 0.5 mM taurine, and 0.01 mM each of the following amino acids; asparagine, glycine, glutamic acid, cysteine, lysine, proline, serine, histidine, and aspartic acid (McKieman et al., Molecular Reproduction and Development 42:188-199, 1995). Typically, the medium also contains commonly used tissue culture antibiotics, such as penicillin and streptomycin. An effective amount of factors are then added daily to either of these base solutions. The term “effective amount” as used herein is the amount of such described factor as to permit a beneficial effect on human PNES cell growth and viability of human PNES cells using judgment common to those of skill in the art of cell culturing and by the teachings supplied herein.

Cell Culture, Maintaining Undifferentiated State and Proliferation

Mouse ES cells can be maintained in a proliferative undifferentiated state in vitro by growing them on feeder layers of MEF cells. An alternative to culturing on feeder layers is the addition of Leukemia inhibitory factor (LIF) to the medium. See Smith, A. G. (2001), Origins and Properties of Mouse Embryonic Stem Cells, Annu. Rev. Cell. Dev. Biol.; Williams, R. L., Hilton, D. J., Pease, S., Wilson, T. A., Stewart, C. L., Gearing, D. P., Wagner, E. F., Metcalf, D., Nicola, N. A., and Gough, N. M. (1998), Myloid Leukemia Inhibitory Factor Maintains the Developmental Potential of Embryonic Stem Cells, Nature. 336, 684-687; Rathjen, P. D., Toth, S., Willis, A., Heath, J. K., and Smith, A. G. (1990) Differentiation Inhibiting Activity is Produced in Matrix-Associated and Diffusible Forms that are Generated by Alternate Promoter Usage, Cell. 62, 1105-1114; Burdon, T, Chambers, I., Stracey, C., Niwa, H., and Smith, A. (1999). Signaling Mechanisms Regulating Self-Renewal and Differentation of Pluripotent Stem Cells. Cells Tissues Organs 165, 131-143; Smith, A. G. (2001). Embryonic stem cells. Marshak, D. R., Gardner, D. K., and Gottlieb, D. eds. (Cold Spring Harbor, N.Y.: Cold Spring Harbor Laboratory Press). 205-230. Those techniques and associated publications are incorporated herein as part of this invention as they are applied to PNES cells. In contrast, even large concentrations of cloned LIF have failed to prevent differentiation of primate ES cell lines in the absence of fibroblast feeder layers. Consequently, we have found that PNES cells and primate ES stem cells are more similar to human EC cells than to mouse pluripotent ES cells, in that they are dependent on the presence of fibroblasts and will not be inhibited from differentiation by LIF in the absence of fibroblasts.

As noted, it has been demonstrated that primate and human pluripotent ES cells will continue to proliferate in vitro in an undifferentiated state within certain culture conditions for longer than one year, and will maintain the developmental potential to contribute to all three embryonic germ layers. See U.S. Pat. No. 6,200,806 and Thompson, J. A. et al. Science, 282:1145-7, 1998. See also Amit, M., Thompson, J. A. et. al. (2000). Clonally Derived Human Embryonic Stem Cell Lines Maintained Pluripotency and Proliferative Potential For Prolonged Periods of Culture. Dev. Biol. 227, 271-278. There are additional methods described in additional publications which allow one to grow pluripotent stem cells in culture indefinitely and in an undifferentiated state, which are also incorporated herein and used to grow PNES cells under such conditions and achieving similar results.

Cryopreservation of PNES Cells

The PNES cells of the present invention for all species may be cryopreserved. Cells, embryos, or portions of animals are routinely frozen and stored at temperatures around −196° C. Cells and embryos can be cryopreserved for an indefinite amount of time. It is known that biological materials can be cryopreserved for more than fifty years and still remain viable. For example, bovine semen that is cryopreserved for more than fifty years can be utilized to artificially inseminate a female bovine animal and result in the birth of a live offspring. There are several programmed freezing protocols that can be used for the purpose of optimization of the survival rate for each particular cell type or each species. Methods and tools for cryopreservation are well-known to those skilled in the art. See, e.g., U.S. Pat. No. 5,160,312, entitled “Cryopreservation Process for Direct Transfer of Embryos,” issued to Voelkel on Nov. 3, 1992.

Alternatively, the human and non-human PNES cells of the present invention may be cryopreserved using the open pulled straw vitrification method. This method is known for the use with embryos and has recently been shown to be very effective for the use with human Pluripotent ES cells. See “Effective cryopreservation of human embryonic stem cells by the open pulled straw vitrification method,” B. E. Reubinoff et al., Human Reproduction, 16:(10) 2187-94 (2001).

The term “thawing” as used herein can refer to a process of increasing the temperature of a cryopreserved cell, embryo, or portions of animals. Methods of thawing cryopreserved materials such that they are active after a thawing process are well-known to those of ordinary skill in the art.

Determining Properties and Characteristics of PNES Cells and PNES Cell Lines

In order to establish that PNES are pluripotent and can proliferate in culture for an indefinite period in an undifferentiated state, we have employed methods and practices similar, and in some cases identical, to those utilized to identify, prove and/or determine the characteristics of animal and human ES and EC cells, which have also displayed the characteristics of pluripotency, undifferentiation and proliferation. Therefore, in order to understand our methods for characterizing the qualities and attributes of PNES, one must have a solid understanding of the development of ES and EC cells in both human and animal models and the different ways in which those cells characteristics and properties have been illustrated or proven.

The mouse has been a very important model for studying pluripotent ES cells and has been a good prototype for generating, identifying and studying human pluripotent ES cells, and therefore proves helpful in defining the characteristics and properties of PNES cells for the purposes of the current invention. For example, it was first demonstrated in the mouse system that pluripotent ES cells can be maintained and propagated in an undifferentiated state (which is important to characterizing PNES cells) provided that the mouse pluripotent ES cells are grown on feeder layer of fibroblast cells (Evans et al., Id.). Recent reports indicate that ES cell lines could be grown in an undifferentiated state without feeder layers by introducing a specific molecule or condition which inhibits differentiation is provided to allow propagation without differentiation (Smith et al., Dev. Biol., 121:1-9 (1987); see also announcements by the Xu, et al. to the effect that it has proliferated ES cell lines without the use of mouse feeder layers by substituting the mouse feeder layers with a mixture of conditioning factors including Matrigel or Laminin and MEF). Because mouse pluripotent ES cells have been shown to be able to proliferate in culture and display pluripotency (see, e.g., Evans et al., Nature, 29:154-156 (1981); Martin, Proc. Natl. Acad. Sci., USA, 78:7634-7638 (1981), the tests and methods used to prove those characteristics and properties are employed with respect to PNES cells. As mentioned above, human EC lines are also pluripotent. As a result of this fact, methods for proving this characteristic and others (e.g., relating to cell morphology, immortality, karyotype, and the expression of certain cell surface markers) are relevant in characterizing PNES cells as being pluripotent in nature.

In addition to mouse pluripotent ES cells and human EC cell lines, since 1998 there have been developments in isolating and studying primate and human pluripotent ES cells. (U.S. Pat. Nos. 5,843,780; 6,200,806; 6,090,622 and Thompson, J. A. et al. Science, 282:1145-7, 1998; M. J. Shamblott et al. Proc. Natl. Acad. Sci. USA, 95:13726-13731, 1998). Since such time it has been found that primate and human pluripotent ES cells display pluripotency, can grow in culture indefinitely in an undifferentiated state, and have normal cell morphology and karyotyping. As a result, the tests applied to human and prmate pluripotent ES cells in an effort to identify these characteristics are relevant under the current invention in characterizing PNES cells.

Stem Cell Morphology

Both mouse and primate pluripotent ES cells have the characteristic morphological features of undifferentiated stem cells, with high nuclear/cytoplasmic ratios, prominent nucleoli, and compact colony formation. PNES cells will display similar colony and cell morphology as the stem cells created/isolated and identified using prior technologies for animal and human pluripotent ES cells. For a broader description of cell morphologies of stem cells, see U.S. Pat. No. 6,200,806 and Thompson, J. A. et al. Science, 282:1145-7, 1998, the texts of which are hereby incorporated by reference.

Cell Surface Markers

Cell surface markers have also been used as supplemental proofs to identify and isolate pluripotent stem cells. There are general cell surface markers used to identify stem cells for all species, and certain cell surface makers used to identify the stem cells for a specific species only. The general cell surface markers provide supplemental proof that PNES cells are in fact stem cells, and the species-specific cell surface markers provide supplemental proof that within that species PNES cells are stem cells.

Available markers: Human and animal pluripotent stem cells are usually characterized by expression of the family of markers comprising the stage-specific embryonic antigens 1-4 (SSEA 1-4), which are cell surface glycolipids that are expressed in early embryonic development and on the surface of pluripotent stem cells. Antibodies recognizing stage-specific embryonic antigens, SSEA 1, SSEA-3 and SSEA-4 are particularly useful in characterizing human and animal stem cells. See NIH Report Stem Cells: Scientific Progress and Future Research Directions, Appendix E Stem Cell Markers (2001), incorporated herein, and available at http://www.nih.gov/news/stemcell/scireport.htm). In addition, antibodies to SSEA 1-4 are available for use in fluorescence activated cell sorting analysis. The antibodies can be obtained from the Developmental Studies Hybridoma Bank of the National Institute of Child Health and Human Development. There are other antigens associated with the extracellular matrix of pluripotent stem cells that are known as surface markers TRA-1-60 and TRA-1-81. (See “Cell Lines from Human Germ Cell Tumors,” In: Robertson E, ed. Teratocarcinomas and Embryonic Stem Cells: A Practical Approach. Oxford: IRL Press, 207-246, 1987). As mentioned, the antibodies used to characterize human ES, EC cells and mouse pluripotent ES cells are also useful in characterizing the PNES cells of the present invention.

Methods for using cell surface markers. In order to detect the presence of stem cell antigens on the surface of the cells, the antibodies are first bound to the cells and subsequently a biotinylated secondary antibody containing an avidin-biotinylated horseradish peroxidase complex is used to detect that an antibody antigen has occurred (Vectastain ABC System, Vector Laboratories).).

Human EC and mouse pluripotent ES cells lines provide important antibody controls for characterizing PNES cells and ES cell lines. Human EC and mouse pluripotent ES cells lines can be distinguished based on the expression of SSEA-1, SSEA-3, SSEA-4, TRA-1-60, and TRA-1-81. In general, pluripotent human EC cell lines are negative for SSEA-1, and are positive for SSEA-3, SSEA-4, TRA-1-60, and TRA-1-81. Therefore, a human EC cell line may be used for comparison with a candidate pluripotent stem cell line. For example, the cell line NTERA-2 cl. D1, is a pluripotent human EC cell line that has been extensively studied and reported in the literature. See Andrews et al., “Cell lines from human germ cell tumors,” In: Robertson E, ed. Teratocarcinomas and Embryonic Stem Cells: A Practical Approach. Oxford: IRL Press, 207-246, 1987. This cell line as well as many other available cell lines may serve as a positive control. In contrast, Mouse ES cells are positive for SSEA-1, and are for negative for SSEA-3, SSEA-4, TRA-1-60, and TRA-1-81. Therefore these cell lines can be used as a negative control for SSEA-3, SSEA-4, TRA-1-60, and TRA-1-81.

The surface expression of certain characteristic stem cell markers on mouse pluripotent ES cells, primate pluripotent ES cells, and human EC cells are shown in Table 1. As is evident from Table 1, primate pluripotent ES cells and human EC cells both express the combination of markers SSEA-3; SSEA-4, TRA-1-60, and TRA-1-81. The glycoproteins SSEA-3 and SSEA-4 are consistently present on human EC cells, and are of diagnostic value in distinguishing human EC cell tumors from human yolk sac carcinomas, choriocarcinomas, and other lineages which lack these markers. See Wenk et al., Int J Cancer 58:108-115, 1994. A recent survey found SSEA-3 and SSEA-4 to be present on all of over 40 human EC cell lines examined (Wenk et al. Int J Cancer 58:108-115, 1994). The antigens known as TRA-1-60 and TRA-1-81 have been well characterized on a particular pluripotent human EC cell line, NTERA-2 CL. D1. See “Cell lines from human germ cell tumors,” In: Robertson E, ed. Teratocarcinomas and Embryonic Stem Cells: A Practical Approach. Oxford: IRL Press, 207-246, 1987. Interestingly, once NTERA-2 CL. D1 cells begin to differentiate in vitro expression of SSEA-3, SSEA-4, TRA-1-60, and TRA-1-81 is lost, while expression of the glycoprotein SSEA-1 is increased. In contrast, undifferentiated mouse pluripotent ES cells express SSEA-1, and do not express SSEA-3 or SSEA-4. A successful PNES cells cell culture prepared according to the present invention will be consistent with the patterns of cell surface markers described in Table 1.

Table 1 shows that human EC cells and human pluripotent ES cells are indistinguishable based on expression of the described markers. Therefore, these two types of cells may be distinguished on the basis of karyotype. As described above, human and primate pluripotent ES cells maintain a normal euploid karyotype while human EC cells are typically aneuploid and thus easily distinguished.

TABLE 1
Marker Expression of ES and EC Cell Lines

| | Stem Cell Lines | | |
|---|---|---|---|
| Marker | Human EC | Mouse ES | Human ES |
| SSEA-1 | Negative | Positive | Negative |
| SSEA-3 | Positive | Negative | Positive |
| SSEA-4 | Positive | Negative | Positive |
| TRA-1-60 | Positive | Negative | Positive |
| TRA-1-81 | Positive | Negative | Positive |

There are several cell surface markers which are used to indicate the characteristics of pluripotent PNES under the current invention including, but not limited to, those found on Table 2.

TABLE 2
PLURIPOTENT STEM CELLS

| Marker Name | Cell Type | Significance |
|---|---|---|
| Alkaline phosphatase | Embryonic stem | Elevated expression of this |
| | (ES), embryonal | enzyme is associated with |
| | carcinoma (EC) | undifferentiated pluripotent |
| | | stem cell (PSC) |
| Alpha-fetoprotein | Endoderm | Protein expressed during |
| (AFP) | | development of primitive |
| | | endoderm; reflects |
| | | endodermal differentiation |
| Bone morphogenetic | Mesoderm | Growth and differentiation |
| protein-4 | | factor expressed during early |
| | | mesoderm formation and |
| | | differentiation |
| Brachyury | Mesoderm | Transcription factor important |
| | | in the earliest phases of |
| | | mesoderm formation and |
| | | differentiation; used as the |
| | | earliest indicator of mesoderm |
| | | formation |
| Cluster designation 30 | ES, EC | Surface receptor molecule |
| (CD30) | | found specifically on PSC |
| Crypto | ES, | Gene for growth factor |
| (TDGF-1) | cardiomyocyte | expressed by ES cells, |
| | | primitive ectoderm, and |
| | | developing cardiomyocyte |
| GATA-4 gene | Endoderm | Expression increases as ES |
| | | differentiates into endoderm |
| GCTM-2 | ES, EC | Antibody to a specific |
| | | extracellular-matrix molecule |
| | | that is synthesized by |
| | | undifferentiated PSCs |
| Genesis | ES, EC | Transcription factor uniquely |
| | | expressed by ES cells either in |
| | | or during the undifferentiated |
| | | state of PSCs |
| Germ cell nuclear | ES, EC | Transcription factor expressed |
| factor | | by PSCs |
| Hepatocyte nuclear | Endoderm | Transcription factor expressed |
| factor-4 (HNF-4) | | early in endoderm formation |
| Nestin | Ectoderm, neural | Intermediate filaments within |
| | and pancreatic | cells; characteristic of |
| | progenitor | primitive neuroectoderm |
| | | formation |
| Nueronal cell-adhesion | Ectoderm | Cell-surface molecule that |
| molecule (N-CAM) | | promotes cell—cell |
| | | interaction; indicates |
| | | primitive neuroectoderm |
| | | formation |
| Oct-4 | ES, EC | Transcription factor unique |
| | | to PSCs; essential for |
| | | establishment and |
| | | maintenance of |
| | | undifferentiated PSCs |
| Pax6 | Ectoderm | Transcription factor expressed |
| | | as ES cell differentiates into |
| | | neuroepithelium |
| Stage-specific | ES, EC | Glycoprotein specifically |
| embryonic antigen-3 | | expressed in early embryonic |
| (SSEA-3) | | development and by |
| | | undifferentiated PSCs |
| Stage-specific | ES, EC | Glycoprotein specifically |
| embryonic antigen-4 | | expressed in early embryonic |
| (SSEA-4) | | development and by |
| | | undifferentiated PSCs |
| Stem cell factor | ES, EC, HSC, | Membrane protein that |
| (SCF or c-kit ligand) | MSC | enhances proliferation of ES |
| | | and EC cells, hematopoietic |
| | | stem cell (HSCs), and |
| | | mesenchymal stem cells |
| | | (MSCs); binds the receptor |
| | | c-kit |
| Telomerase | ES, EC | An enzyme uniquely |
| | | associated with immortal cell |
| | | lines; useful for identifying |
| | | undifferentiated PSCs |
| TRA-1-60 | ES, EC | Antibody to a specific |
| | | extracellular matrix molecule |
| | | is synthesized by |
| | | undifferentiated PSCs |
| TRA-1-81 | ES, EC | Antibody to a specific |
| | | extracellular matrix molecule |
| | | normally synthesized by |
| | | undifferentiated PSCs |
| Vimentin | Ectoderm, neural | Intermediate filaments within |
| | and pancreatic | cells; characteristic of |
| | progenitor | primitive neuroectoderm |
| | | formation |

Application of cell markers to PNES cells. The PNES cells of the present invention are positive for alkaline phosphatase, similar to the situation found with pluripotent ES cells. For example, pluripotent ES cells all are known to express alkaline phosphatase and monitoring this enzyme can be useful during the isolation, culturing and characterization of these cells. The expression of alkaline phosphatase is shared by both primate and mouse pluripotent ES cells, and relatively few other embryonic cells express this enzyme. Positive cells include the ICM and primitive ectoderm (which are the most similar embryonic cells in the intact embryo to pluripotent ES cells), germ cells (which are totipotent), and a very limited number of neural precursors. See Kaufman M H. The atlas of mouse development. London: Academic Press, 1992.

Pluripotency

Pluripotency has been proven by injecting candidate ES cells into mice with severe combined immunodeficiency (SCID) and analyzing the cell types comprising the resulting tumors, which have been shown to differentiate into cells representing all three germ layers. All selected PNES cell lines are injected into mice with SCID and are able to differentiate into cells representing all three germ layers. For example, approximately 0.5-1.0×10⁶ candidate PNES cells are injected into the rear leg muscles or testis of 8-12 week old male SCID mice (6-10 mice) and let grow until forming the tumor-like cell mass. The resulting tumors are fixed in 4% paraformaldehyde and examined histologically after paraffin embedding at 8-16 weeks of development. Next, the embedded tumors are sectioned and cell types comprising the tumor are evaluated. In the preferred embodiment, PNES cells demonstrate the ability to differentiate into the following: cartilage, smooth muscle, and striated muscle (mesoderm); stratified squamous epithelium with hair follicles, neural tube with ventricular, intermediate, and mantle layers (ectoderm); ciliated columnar epithelium and villi lined by absorptive enterocytes and mucus-secreting goblet cells (endoderm). It should be noted that these are only a few of the cell types that may be present in the tumors and this list is not meant to be exhaustive.

Multiple techniques for proving pluripotency for mouse ES cells re described in Smith A. G. (2001), Origins and Properties of Mouse Embryonic Stem Cells, Annu. Rev. Cell. Dev. Biol., which such report and techniques/methods are incorporated herein and is used under the current invention to prove pluripotency. These methods include methods similar to that described above, and also a technique under which the feeder layers are removed and leukemia inhibitory factor (LIF) is added to the growth medium, and within a few days of changing the culture conditions, pluripotent cells (PNES cells or ES cells) aggregate and may form embryoid bodies (EB) which consist of cells which are both differentiated and partially differentiated that are derived from the three primary germ layers.

Karyotype

The present invention provides human and animal PNES cells that have normal karyotypes, similar to what has been seen in other stem cells (human and nonhuman ES lines). In addition, both XX and XY cells lines will be derived. A normal karyotype indicates that all chromosomes normally characteristic of the species are present and have not been noticeably altered. Cell lines can be karyotyped with a standard G-banding technique (such as by the Cytogenetics Laboratory of the University of Wisconsin State Hygiene Laboratory, which provides routine karyotyping services) and compared to published karyotypes for the primate species.

A karyotype is the particular chromosome complement of an individual or of a related group of individuals, as defined both by the number and morphology of the chromosomes usually in mitotic metaphase. It includes such things as total chromosome number, copy number of individual chromosome types (e.g., the number of copies of chromosome X), and chromosomal morphology, e.g., as measured by length, centromeric index, connectedness, or the like. Chromosomal abnormalities can be detected by examination of karyotypes. Karyotypes are conventionally determined by staining a cell's metaphase, or otherwise condensed (for example, by premature chromosome condensation) chromosomes.

A number of cytological techniques based upon chemical stains have been developed which produce longitudinal patterns on condensed chromosomes, generally referred to as bands. The banding pattern of each chromosome within an organism usually permits unambiguous identification of each chromosome type, Latt, “Optical Studies of Metaphase Chromosome Organization,” Annual Review of Biophysics and Bioengineering Vol. 5, pgs. 1-37 (1976). Accurate detection of some important chromosomal abnormalities, such as translocations and inversions, has required such banding analysis.

Immortality

The PNES cells of the present invention are immortal. Immortal cells are capable of continuous indefinite replication in vitro. As a practical matter, immortality is measured by observing continued proliferation of cells for longer than one year in culture. Likewise, primary cell cultures that are not immortal fail to continuously divide for this length of time. See Freshney, Culture of animal cells. New York: Wiley-Liss, 1994. It has been demonstrated that primate and human pluripotent ES cells will continue to proliferate in vitro with the culture conditions described below for longer than one year, and will maintain the developmental potential to contribute to all three embryonic germ layers. See U.S. Pat. No. 6,200,806 and Thompson, J. A. et al. Science, 282:1145-7, 1998. The methods described and utilized by Thompson are incorporated herein by reference as one of the methods deployed under the current invention to grow PNES in vitro for an indefinite period and in an undifferentiated state. Note that to date, it has not been demonstrated that the pluripotent stem cells generated from embryonic germ cells have this property. U.S. Pat. No. 6,090,622 and M. J. Shamblott et al. Proc. Natl. Acad. Sci. USA, 95:13726-13731, 1998.

Whether a candidate PNES cell line has retained the proper developmental potential along with its immortality can be determined by injecting the PNES cell lines into SCID mice after being grown and maintained in culture for one year. In the preferred embodiment, the PNES cell lines are cultured for the time period in question, usually 1 year, and then about 0.5-1.0×10⁶ candidate PNES cells are injected into the rear leg muscles or testis of 8-12 week old male SCID mice (6-10 mice). The resulting tumors can be fixed in 4% paraformaldehyde and examined histologically after paraffin embedding at 8-16 weeks of development. It is possible that karyotypic changes can occur randomly in some cells with prolonged culture, however some PNES cells will maintain a normal karyotype for longer than a year of continuous culture as proven by the tests for karyotyping described above.

Multipotent/Adult Stem Cells (ASC's) and Specific Differentiated Cells

Directing Differentation of Pluripotent PNES to ASC's and Specific Differentiated Cells. There are various and differing techniques and methods for directing PNES cells to become different types of ASC's and Specific Differentiated Cells in vitro, including, but not limited to, into the following cell types: adipocyte, astrocyte, cardiomyocyte, chondrocyte, definitive hematopoietic, dendritic, endothelial, keratinocyte, lymphoid precursor, mast, neuron, oligodendrocyte, osteoblast, pancreatic islets, primitive hematopoietic, smooth muscle, striated muscle, yolk sac endoderm, and yolk sac mesoderm. As evidenced, these techniques can be utilized to direct pluripotent human cells such as PNES into cells derived from all three germ layers, and publications describing those techniques cited here and the relevant techniques described therein are incorporated completely under the current invention and are used to prove similar results with respect to PNES and derivatives thereof. Kehar, I., Kenyagin-Karsenti, D., Druckmann, M., Segev, H., Amit, M., Gepstein, A., Livne, E., Binah, O., Itskovitz-Eldor, J., and Gepstein, L. (2001). Human ES cells can differentiate into myocytes portraying cardiomyocytic structural and functional properties. J. Clin. Invest. (In press); Itskovitz-Eldor, J., Schuldiner, M., Karsenti, D., Eden, A., Yanuka, O., Amit, M., Soreq, H., and Benvenisty, N. (2000). Differentiation of human embryonic stem cells into embryoid bodies comprising the three embryonic germ layers. Mol. Med. 6, 88-95; Assady, S., Maor, G., Amit, M., Itskovitz-Eldor, J., Skorecki, K. L., and Tzukerman, M. (2001). Insulin production by human embryonic stem cells. Diabetes, 50; and Kerr, D. A., Llado, J., Shamblott, M., Maragakis, N., Irani, D. N., Dike, S., Sappington, A., Gearhart, J., and Rothstein, J. (2001). Human embryonic germ cell derivatives facilitate motor recovery of rats with diffuse motor neuron injury.

Some additional specific examples include methods for directing pluripotent human stem cells into bone, cartilage, squamouos and cuboidal epithelium, neural cells, grandular epithelium and striated muscle, and the techniques relating to directing PNES cells into those particular types of cells as described in the following citations are also incorporated completely under the current invention and are used to prove similar results with respect to PNES cells and derivatives thereof. See Reubinoff, B. E., Pera, M. F., Fong, C. Y., Trounson, A., and Bongso, A. (2000). Embryonic stem cell lines from human blastocysts: somatic differentiation in vitro. Nat. Biotechnol. 18, 399-404; and Roach, S., Cooper, S., Bennett, W., and Pera, M. F. (1993). Cultured cell lines from human teratomas: windows into tumor growth and differentiation and early human development. Eur. Uro. 23, 82-87. In general terms, to aid in understanding the underlying techniques themselves, the methods for directing pluripotent stem cells to become ASC's and Specific Differentiated Cells include, but are not limited to, (a) adding growth factors to the culture medium or changing the chemical composition of the surface on which the pluripotent cells are growing, and (b) introducing foreign genes into the pluripotent cells via transfection or other methods, the result of which is to add an active gene to the pluripotent cell genome which then triggers the cells to differentiate along a particular pathway, c) co-culturing with inactivated primary specialized cells or tissues, or in the presence of those tissue matrix components, d) using media supplemented with the extracts prepared from the specialized tissues and/or organs.

The techniques and methods of differentiation described in the following publications and the publications cited therein are herein incorporated by reference in their entirety under the current invention and are used to provide similar results with respect to PNES cells and derivatives thereof.

Adipocyte

Dani, C., Smith, A. G., Dessolin, S., Leroy, P., Staccini, L., Villageois, P., Darimont, C., and Ailhaud, G. (1997). Differentiation of embryonic stem cells into adipocytes in vitro. J. Cell. Sci. 110, 1279-1285.

Atrocyte

Fraichard, A., chassandre, O., bilbaut, G., Dehay, C., Savatier, P., and Samarut, J. (1995). In vitro differentiation of embryonic stem cells into glial cells and functional neurons.

Cardiomyocyte

Doetschman, T., Eistetter, H., Katz, M., Schmit, w., and Kemler, R. (1985). The in vitro development of blastocysts-derived embryonic stem cell lines: formatoin of visceral yolk sac, blood islands and myocardium. J. Embryol. Exp. Morph. 87, 27-45.

Maltsev, V. A., rohwedel, J., Hescheler, J., and Wobus, A. M. (1993). Embryonic stem cells differentiate in vitro into cardiomyocytes representing sinusnodal, atrial and ventricular cell types. Mech. Dev. 44,41-50.

Chondrocyte

Kramer, J., Hegert, C., Guan, K., Wobus, A. M., Muller, P. K., and Rohwedel, J. (2000). Embryonic stem cell-derived chondrogenic differentiation in vitro: activation by BMP-2 and BMP-4. Mech. Dev. 92, 193-205.

Definitive Hematopoietic

Nakano, T., Kodama, H., and Honjo, T. (1996). In vitro development of primitive and definitive erythrocytes from different precursors. Science. 272, 722-724.

Nishikawa, S., Hirashima, M., Matsuyoshi, N., and Kodama, H. (1998). Progressive lineage analysis by cell sorting and culture identifies FLK1(+)VE-cadherin(+) cells at a diverging point of endothelial and hemopoietic lineages. Development. 125, 1747-1757.

Wiles, M. V. and Keller, G. (1991). Multiple hematopoietic lineages develop from embryonic stem (ES) cells in culture. Development. 111, 259-267.

Dendritic Cell

Fairvhild, P. J., Brook, F. A., Gardner, R. L., Graca, L., Strong, V., Tone, Y., Tone, M., Nolan, K. F., and Waldmann, H. (2000). Directed differentiation of dendritic cells from mouse embryonic stem cells. Curr. Biol. 10, 1515-1518.

Endothelial Cell

Risau, W., Sarioloa, H., Zerwes, H. G., Sasse, J., Ekblom, P., Kemler, R., and Doetschman, T. (1988). Vasculogenesis and angiogenesis in embryhonic-stem-cell-derived embryoid bodies. Development. 102, 471-478.

Yamashita, J., Itoh, H., Hirashima, M., Ogawa, M., Nishikawa, S., Yurugi, T., Naito, M., Nakao, K., and Nishikawa, S. (2000). Flk1-positive cells derived from embryonic stem cells serve as vascular progenitors. Nature. 408, 92-96.

Keratinocyte

Bagutti, C., Wobus, A. M., Fassler, r., and Watt, f. M. (1996). Differentiation of embryonal stem cells into keratinocytes: comparison of wild-type and B(1) integrin-deficient cells. Dev. Biol. 179, 184-196.

Yamashita, J., Itoh, H., Hirashima, M., Ogawa, M., Nishikawa, S., Yurugi, T., Naito, M., Nakao, K., and Nishikawa, S. (2000). Flk1-positive cells derived from embryonic stem cells serve as vascular progenitors. Nature. 408, 92-96.

Lymphoid Precursor

Potocnik, A. J., Nielsen, P. J., and Eichmann, K. (1994). In vitro generation of lymphoid precursors from embryonic stem cells. EMBO. J. 13, 5274-5283.

Mast Cell

Tsai, M., Wedemeyer, J., Ganiatsas, S., Tam, S. Y., Zon, L. I., and Galli, S. J. (2000). In vivo immunological function of mast cells derived from embryonic stem cells: an approach for the rapid analysis of even embryonic lethal mutations in adult mice in vivo. Proc. Natl. Acad. Sci. U.S.A. 97, 9186-9190.

Neuron

Bain, G., Kitchens, d., Yao, M., Huettner, J. E., and Gottlieb, D. I. (1995). Embryonic stem cells express neuronal properties in vitro. Dev. Biol. 168, 342-357.

Strubing, C., Ahnert-Hilger, G., Shan, J., Wiedenmann, B., Hescheler, J., and Wobus, A. M. (1995). Differentiation of pluripotent embryonic stem cells into the neuronal lineage in vitro gives rise to mature inhibitory and excitatory neurons. Mech. Dev. 53, 275-287.

Oligodendrocyte

Brustle, O., Jones, K. N., Learish, R. D., Karram, K., Choudhary, K., Wiestler, O. D., Duncan, I. D., and McKay, R. D. (1999). Embryonic stem cell-derived glial precursors: a source of myelinating transplants. Science. 285, 754-756.

Liu, S., Qu, Y., Stewart, T. J., Howard, M. J., Chakrabortty, S., Holekamp, T. F., and Mcdonald, J. W. (2000). Embryonic stem cells differentiate into oligodendrocytes and myelinate in culture and after spinal cord transplantation. Proc. Natl. Acad. Sci. U.S.A. 97, 6126-6131.

Ostenblast

Buttery, L. D., Borne, S., Xynos, J. D., Wood, H., Hughes, F. J., Hughes, S. P., Episkopou, V., and Polak, J. M. (2001). Differentiation of osteoblasts and in vitro bone formation from murine embryonic stem cells. Tissue Eng. 7, 89-99.

Pancreatic Islets

Lumelsky, N., Biondel, O., Laeng, P., Velasco, I., Ravin, R., and McKay, R. (2001). Differentiation of Embryonic Stem Cells to Insulin-Secreting Structures Similar to Pancreatic Islets. Science. 292, 1389-1394.

Primitive Hematopoiectic

Doetschman, T., Eistetter, H., Katz, M., Schmit, w., and Kemler, R. (1985). The in vitro development of blastocysts-derived embryonic stem cell lines: formatoin of visceral yolk sac, blood islands and myocardium. J. Embryol. Exp. Morph. 87, 27-45.

Nakano, T., Kodama, H., and Honjo, T. (1996). In vitro development of primitive and definitive erythrocytes from different precursors. Science. 272, 722-724.

Smooth Muscle

Yamashita, J., Itoh, H., Hirashima, M., Ogawa, M., Nishikawa, S., Yurugi, T., Naito, M., Nakao, K., and Nishikawa, S. (2000). Flk1-positive cells derived from embryonic stem cells serve as vascular progenitors. Nature. 408, 92-96.

Stirated Muscle

Rohwedel, J., Maltsev, V., Bober, e., Arnold, J. J., Hescheler, J., and Wobus, A. M. (1994). Muscle cell differentiation of embryonic stem cells reflects myogenesis in vivo: developmentally regulated expression of myogenic determination genes and functional expression of ionic currents. Dev. Biol. 164, 87-101.

Yolk Sac Endoderm

Doetschman, T., Eistetter, H., Katz, M., Schmit, w., and Kemler, R. (1985). The in vitro development of blastocysts-derived embryonic stem cell lines: formatoin of visceral yolk sac, blood islands and myocardium. J. Embryol. Exp. Morph. 87, 27-45.

Yolk Sac Mesoderm

Doetschman, T., Eistetter, H., Katz, M., Schmit, w., and Kemler, R. (1985). The in vitro development of blastocysts-derived embryonic stem cell lines: formatoin of visceral yolk sac, blood islands and myocardium. J. Embryol. Exp. Morph. 87, 27-45.

Cell surface markers. There are various cell surface markers employed under the current invention to isolate, identify and define the characteristics of the ASC's and/or Specific Differentiated Cells created under the current invention including, but not limited to, those described on Table 3 which are incorporated herein.

TABLE 3
MARKERS COMMONLY USED TO IDENTIFY
ADULT STEM CELLS AND TO CHARACTERIZE
DIFFERENTIATED OR SPECIFIC CELL TYPES

| | | |
|---|---|---|
| Marker Name | Cell Type | Significance |

BLOOD VESSELL

| | | |
|---|---|---|
| Fetal liver kinase-1 | Endothelial | Cell-surface receptor protein |
| (Flk1) | | that identifies endothelial cell |
| | | progenitor; marker of |
| | | cell—cell contacts |
| Smooth muscle cell- | Smooth muscle | Identifies smooth muscle cells |
| specific myosin | | in the wall of blood vessels |
| heavy chain | | |
| Vascular endothelial | Smooth muscle | Identifies smooth muscle cells |
| cell cadherin | | in the wall of blood vessels |

BONE

| | | |
|---|---|---|
| Bone-specific | Osteoblast | Enzyme expressed in |
| alkaline phosphatase | | osteoblast; activity indicates |
| (BAP) | | bone formation |
| Hydroxyapatite | Osteoblast | Mineralized bone matrix that |
| | | provides structural integrity; |
| | | marker of bone formation |
| Osteocalcin | Osteoblast | Mineral-binding protein |
| (OC) | | uniquely synthesized by |
| | | osteoblast; marker of bone |
| | | formation |

BONE MARROW AND BLOOD

| | | |
|---|---|---|
| Bone morphogenetic | Mesenchymal | Important for the |
| protein receptor | stem and | differentiation of committed |
| (BMPR) | progenitor | mesenchymal cell types from |
| | cells | the mesenchymal stem and |
| | | progenitor cells; BMPR |
| | | identifies early mesenchymal |
| | | lineages (stem and progenitor |
| | | cells) |
| CD4 and CD8 | White blood cell | Cell-surface protein markers |
| | (WBC) | specific for mature T |
| | | lymphocyte (WBC subtype) |
| CD34 | Hematopoietic | Cell-surface protein on bone |
| | stem cell (HSC), | marrow cell, indicative of |
| | satellite, | a HSC and endothelial |
| | endothelial | progenitor; CD34 also |
| | progenitor | identifies muscle satellite, |
| | | a muscle stem cell |
| CD34 + Scal + Lin- | Mesencyhmal | Identifies MSCs, which can |
| profile | stem cell (MSC) | differentiate into adipocyte, |
| | | osteocyte, chondrocyte, and |
| | | myocyte |
| CD38 | Absent on HSC | Cell-surfaced molecule that |
| | Present on WBC | identifies WBC lineages,. |
| | lineages | Selection of CD34+/CD38− |
| | | cells allows for purification of |
| | | HSC populations |
| CD44 | Mesenchymal | A type of cell-adhesion |
| | | molecule used to identify |
| | | specific types of mesenchymal |
| | | cells |
| c-Kit | HSC, MSC | Cell-surface receptor on BM |
| | | cell types that identifies HSC |
| | | and MSC; binding by fetal |
| | | calf serum (FCS) enhances |
| | | proliferation of ES cells, |
| | | HSCs, MSCs, and hemato- |
| | | poietic progenitor cells |
| Colony-forming unit | HSC, MSC | CFU assay detects the ability |
| (CFU) | progenitor | of a single stem cell or |
| | | progenitor cell to give rise to |
| | | one or more cell lineages, |
| | | such as red blood cell (RBC) |
| | | and/or white blood cell |
| | | (WBC) lineages |
| Fibroblast colony- | Bone marrow | An individual bone marrow |
| forming unit (CFU-F) | fibroblast | cell that has given rise to a |
| | | colony of multipotent |
| | | fibroblastic cells; such |
| | | identified cells are precursors |
| | | of differentiated mesenchymal |
| | | lineages |
| Hoechst dye | Absent on HSC | Fluorescent dye that bind |
| | | DNA; HSC extrudes the dye |
| | | and stains lightly compared |
| | | with other cell types |
| Leukocyte common | WBC | Cell-surface protein on WBC |
| antigen (CD45) | | progenitor |
| Lineage surface | HSC, MSC | Thirteen to 14 different cell- |
| antigen (Lin) | Differentiated | surface proteins that are |
| | RBC and WBC | markers of mature blood cell |
| | lineages | lineages; detection of Lin- |
| | | negative cells assists in the |
| | | purification of HSC and |
| | | hematopoietic progenitor |
| | | populations |
| Mac-1 | WBC | Cell-surface protein specific |
| | | for mature granulocyte and |
| | | macrophage (WBC subtypes) |
| Muc-18 (CD146) | Bone marrow | Cell-protein (immunoglobulin |
| | fibroblasts, | superfamily) found on bone |
| | endothelial | marrow fibroblasts, which |
| | | may be important in |
| | | hematopoiesis; a |
| | | subpopulation of Muc-18+ |
| | | cells are mesenchymal |
| | | precursors |
| Stem cell antigen | HSC, MSC | Cell-surface protein on bone |
| (Sca-1) | | marrow (BM) cell, indicative |
| | | of HSC and MSC |
| Stro-1 antigen | Stromal | Cell-surface glycoprotein on |
| | (mesenchymal) | subsets of bone marrow |
| | precursor cells, | stromal (mesenchymal) cells; |
| | hematopoietic | selection of Stro-1+ cells |
| | cells | assists in isolating |
| | | mesenchymal precursor cells, |
| | | which are multipotent cells |
| | | that give rise to adipocyte, |
| | | osteocyte, smooth myocyte, |
| | | fibroblasts, chondrocyte, and |
| | | blood cells |
| Thy-1 | HSC, MSC | Cell-surface protein; negative |
| | | or low detection is suggestive |
| | | of HSC |

CARTILAGE

| | | |
|---|---|---|
| Collagen types II IV | Chondrocyte | Structural proteins produced |
| | | specifically by chondrocyte |
| Keratin | Keratinocyte | Principal protein of skin; |
| | | identifies differentiated |
| | | keratinocyte |
| Sulfated | Chondrocyte | Molecule found in connective |
| proteoglycan | | tissues; synthesized by |
| | | chondrocyte |

FAT

| | | |
|---|---|---|
| Adipocyte lipid- | Adipocyte | Lipid-binding protein located |
| binding protein | | specifically in adipocyte |
| (ALBP) | | |
| Fatty acid | Adipocyte | Transport molecule located |
| transporter | | specifically in adipocyte |
| (FAT) | | |
| Adipocyte lipid- | Adipocyte | Lipid-binding protein located |
| binding protein | | specifically in adipocyte |
| (ALBP) | | |

GENERAL

| | | |
|---|---|---|
| Y chromosome | Male cells | Male-specific chromosome |
| | | used in labeling and detecting |
| | | donor cells in female |
| | | transplant recipients |
| Karyotype | Most cell types | Analysis of chromosome |
| | | structure and number in a cell |

LIVER

| | | |
|---|---|---|
| Albumin | Hepatocyte | Principal protein produced by |
| | | the liver; indicates functioning |
| | | of maturing and fully |
| | | differentiated hepatocytes |
| B-1 integrin | Hepatocyte | Cell-adhesion molecule |
| | | important in cell—cell |
| | | interactions; marker expressed |
| | | during development of liver |

NERVOUS SYSTEM

| | | |
|---|---|---|
| CD133 | Neural stem cell, | Cell-surface protein that |
| | HSC | identifies neural stem cells, |
| | | which give rise to neurons and |
| | | glial cells |
| Glial fibrillary acidic | Astrocyte | Protein specifically produced |
| protein (GFAP) | | by astrocyte |
| Microtubule- | Neuron | Dendrite-specific MAP; |
| associated protein-2 | | protein found specifically in |
| (MAP-2) | | dendritic branching of neuron |
| Myelin basic protein | Oligodendrocyte | Protein produced by mature |
| (MPB) | | oligodendrocytes; located in |
| | | the myelin sheath surrounding |
| | | neuronal structures |
| Nestin | Neural | Intermediate filament |
| | progenitor | structural protein expressed |
| | | in primitive neural tissue |
| Neural tubulin | Neuron | Important structural protein |
| | | for neuron; identifies |
| | | differentiated neuron |
| Neurofilament | Neuron | Important structural protein |
| (NF) | | for neuron; identifies |
| | | differentiated neuron |
| Neurosphere | Embryoid body | Cluster of primitive neural |
| | (EB), ES′ | cells in culture of |
| | | differentiating ES cells; |
| | | indicates presence of early |
| | | neurons and glia |
| Noggin | Neuron | A neuron-specific gene |
| | | expressed during the |
| | | development of neurons |
| O4 | Oligodendrocyte | Cell-surface marker on |
| | | immature, developing |
| | | oligodendrocyte |
| O1 | Oligodendrocyte | Cell-surface marker that |
| | | characterizes mature |
| | | oligodendrocyte |
| Synaptophysin | Neuron | Neuronal protein located in |
| | | synapses; indicates |
| | | connections between neurons |
| Tau | Neuron | Type of MAP; helps maintain |
| | | structure of the axon |

PANCREAS

| | | |
|---|---|---|
| Cytokeratin 19 | Pancreatic | CK19 identifies specific |
| (CK19) | epithelium | pancreatic epithelial cells that |
| | | are progenitors for islet cells |
| | | and ductal cells |
| Glycogen | Pancreatic islet | Expressed by alpha-islet cell |
| | | of pancreas |
| Insulin | Pancreatic islet | Expressed by beta-islet cell of |
| | | pancreas |
| Insulin-promoting | Pancreatic islet | Transcription factor expressed |
| factor-1 (PAX-1) | | by beta-islet cell of pancreas |
| Nestin | Pancreatic | Structural filament protein |
| | progenitor | indicative of progenitor cell |
| | | lines including pancreatic |
| Pancreatic | Pancreatic islet | Expressed by gamma-islet cell |
| polypeptide | | of pancreas |
| Somatostatin | Pancreatic islet | Expressed by delta-islet cell |
| | | of pancreas |

SKELETAL MUSCLE/CARDIAC/SMOOTH MUSCLE

| | | |
|---|---|---|
| MyoD and Pax7 | Myoblast, | Transcription factors that |
| | Myocyte | direct differentiation of |
| | | myoblasts into mature |
| | | myocytes |
| Myogenin and MR4 | Skeletal myocyte | Secondary transcription |
| | | factors required for |
| | | differentiation of myoblasts |
| | | from muscle stem cells |
| Myosin heavy chain | Cardiomyocyte | A component of structural and |
| | | contractile protein found in |
| | | cardiomyocyte |
| Myosin light chain | Skeletal myocyte | A component structural and |
| | | contractile protein found in |
| | | skeletal myocyte |

Summary—Isolation and Differentiation of PNES Cells, ASC's and Specific Differentiated Cells

As indicated throughout this detailed discussion, there are many techniques and methods for isolating, identifying, differentiation and directing PNES cells, ASC's and Specific Differentiated Cells. Many of these techniques are summarized in the following references, which are hereby incorporated by reference in their entirety methods of performing these tasks under the current invention.

1. Alison, M. R., Poulsom, r., Jeffery, r., Dhillon, A. P., Quaglia, A., Jacob, J., Novelli, M., Prentice, G., Williamson, J., and Wright, N. A. (2000). Hepatocytes from non-hepatic adult stem cells. Nature. 406, 257.

2. Andrews, P. W., Damjanov, I., Simon, D., Banting, G. S., Carlin, C., Dracopoli, N. C., and Fogh, J. (1984). Pluripotent embryonal carcinoma clones derived from the human teratocarcinoma cell line Tera-2. Differentiation in vivo and in vitro. Lab. Invest. 50, 147-162.

3. Assady, S., Maor, G., Amit, M., Itskovitz-Eldor, J., Skorecki, K. L., and Tzukerman, M. (2001). Insulin production by human embryonic stem cells. Diabetes, 50, http://www.diabetes.org/Diabetes_Rapids/Suheir_Assady_{—}06282001.pdf.

4. Azizi, S. A., Stokes, D., Augelli, B. J., DiGirolamo, C., and Prockop, D. J. (1998). Engraftment and migration of human bone marrow stromal cells implanted in the brains of albino rats-similarities to astrocyte grafts. Proc. Natl. Acad. Sci. U.S.A. 95, 3908-3913.

5. Bader, A., Al Dubai, H., and Weitzer, G. (2000). Leukemia inhibitory factor modulates cardiogenesis in embryoid bodies in opposite fashions. Circ. Res. 86, 787-794.

6. Bagutti, C., Wobus, A. M., Fassler, r., and Watt, f. M. (1996). Differentiation of embryonal stem cells into keratinocytes: comparison of wild-type and B(1) integrin-deficient cells. Dev. Biol. 179, 184-196.

7. Dani, C., Smith, A. G., Dessolin, S., Leroy, P., Staccini, L., Villageois, P., Darimont, C., and Ailhaud, G. (1997). Differentiation of embryonic stem cells into adipocytes in vitro. J. Cel. Sci. 110, 1279-1285.

8. Baum, C. M., Weissman, I. L., Tsukamoto, A. S., Buckle, A. M., and Peault, B. (1992). Isolation of a candidate human hematopoietic stem-cell population. Proc. Natl. Acad. Sci. U.S.A. 89, 2804-2808.

9. Bittner, R. E., Schofer, C., Weipoltshammer, K., Ivanova, S., Streubel, B., Hauser, E., Freilinger, M., Hoger, H., Eble-Burger, A., and Wachtler, F. (1999). Recruitment of bone-marrow-derived cells by skeletal and cardiac muscle in adult dystrophic mdx mice. Anat, Embryol. (Berl) 199, 391-396.

10. Bjornson, c. R., Rietze, R. L., Reynolds, B. A., Magli, M. C., and Vescovi, A. L. (1999). Turning brain into blood: a hematopoietic fate adopted by adult neural stem cells in vivo. Science. 283, 534-537.

11. Bosch, P., Musgrave, D. S., Lee, J. Y., Cummins, J., Shuler, F., Ghivizzani, S. C., Evans, C., Robbins, P. D., and Huard, J. (2000). Osteoprogenitor cells within skeletal muscle. J. Orthop. Res. 18, 933-944.

12. Brazelton, T. R., Rossi, F. M., Keshet, G. I., and Blau, H. M. (2000). From marrow to brain: expression of neuronal phenotypes in adult mice. Science. 290, 1775-1779.

13. Broxmeyer, H. E., Douglas, G. W., Hangoc, G., Cooper, S., Bard, J., English, d., Arny, M., Thomas, L., and Boyse, E. A. (1989). Human unbilical cord blood as a potential source of transplantable hematopoietic sterm/progenitor cells. Proc. Natl. Acad. Sci. U.S.A. 86, 3828-3832.

14. Brustle, O., Jones, K. N., Learish, R. D., Karram, K., Choudhary, K., Wiestler, O. D., Duncan, I. D., and McKay, R. D. (1999). Embryonic stem cell-derived glial precursors: a source of myelinating transplants. Science. 285, 754-756.

15. Buttery, L. D., Bourne, S., Xynos, J. D., Wood, H., Hughes, F. J., Hughes, S. P., Episkopou, V., and Polak, J. M. (2001). Differentiation of osteoblasts and in vitro bone formation from murine embryonic stem cells. Tissue Eng. 7, 89-99.

16. Dani, C., Smith, A. G., Dessolin, S., Leroy, P., Staccini, L., Villageois, P., Darimont, C., and Ailhaud, G. (1997). Differentiation of embryonic stem cells into adipocytes in vitro. J. Cel. Sci. 110, 1279-1285.

17. Dinsmore, J., Ratliff, J., Deacon, S., Pakzaban, P., Jacoby, D., Galpern, W., and Isacson, O. (1996). Embryonic stem cells differentiated in vitro as a novel source of cells for transplantation. Cell Transplant. 5, 131-143.

18. Doetsch, F., Caille, I., Lim. D. A., Garcia-Verdugo, J. M., and Alvarez-Buylia, A. (1999). Subventricular zone astrocytes are neural stem cells in the adult mammalian brain. Cell. 97, 703-716.

19. Doetschman, T., Eistetter, H., Katz, M., Schmit, w., and Kemler, R. (1985). The in vitro development of blastocysts-derived embryonic stem cell lines: formatoin of visceral yolk sac, blood islands and myocardium. J. Embryol. Exp. Morph. 87, 27-45.

20. Drab, M., Haller, H., Bychkov, R., Erdmann, B., Lindschau, C., Haase, H., Morano, I., Luft, F. C., and Wobus, A. M. (1997). From totipotent embryonic stem cells to spontaneously contracting smooth muscle cells: a retinoic acid and db-cAMP in vitro differentiation model. FASEB J. 11, 905-915.

21. Eglitis, M. A., and Mezey, e. 91997). Hematopoietic cells differentiate into both microglia and macroglia in the brains of adult mice. Proc. Natl. Acad. Sci. U.S.A. 94, 4080-4085.

22. Erices, A., Conget, P., and Minguell, J. J. (1999). Mesenchymal progenitor cells in human unbilical cord blood. Br. J. Haematol. 109, 235-242.

23. Evans, M. J. and Kaufman, M. H. (1981). Establishment in culture of pluripotential cells from mouse embryos. Nature. 292, 154-156.

24. Fairvhild, P. J., Brook, F. A., Gardner, R. L., Graca, L., Strong, V., Tone, Y., Tone, M., Nolan, K. F., and Waldmann, H. (2000). Directed differentiation of dendritic cells from mouse embryonic stem cells. Curr. Biol. 10, 1515-1518.

25. Ferrari, G., Cusella-De Angelis, G., Coletta, M., Paolucci, E., Stomaiuolo, A., Cossu, G., and Mavillo, F. (1998). Muscle regeneration by bone marrow-derived myogenic progenitors. Science. 279, 1528-1530.

26. Fraichard, A., chassandre, O., bilbaut, G., Dehay, C., Savatier, P., and Samarut, J. (1995). In vitro differentiation of embryonic stem cells into glial cells and functional neurons. J. Cell sci. 108, 3181-3188.

27. Friedenstein, A. J., Gorskaja, U. F., and Kulagina, N. N. (1976). Fibroblast presursor in normal and irradiated mouse hematopoietic organs. Exp. Hematol. 4, 267-274.

28. Galli, R., Borello, U., gritti, A., Minasi, M. G., Bjornson, C., Coletta, M., Mora, M., De Angelis, M. G., Fiocco, R., Cossu, g., and Vescovi., A. L. (2000). Skeletal myogenic ptential of human and mouse neural stem cells. Nat. Neurosci. 3, 986-991.

29. Gottlieb, D. I. and Huettner, J. E. (1999). An in vitro pathway from embryonic stem cells to neurons and glia. Cells Tissues Organs. 165, 165-172.

30. Grimaldi, P. A., Teboul, L., Inadera, H., Gaillard, D., and Amri, E.Z. (1997). Trans-differentiation of myoblasts to adipoblasts: Prostaglandins. Leukot. Esent. Fatty Acids. 57, 71-75.

31. Gussoni, e., soneoka, Y., Strickland, C. D., buzney, E. A., Khan, M. K., Flint, A. F., Kunkel, L. M. and Mulligan, R. C. (1999). Dystrophin expression in the mdx mouse restored by stem cell transplantation. Nature. 401, 390-394.

32. Hirashima, M., Kataoka, H., Nishikawa, S., Matsuyoshi, N., and Nishikawa, S. (1999). Maturation of embryonic stem cells into endothelial cells in an in vitro model of vasculogenesis. Blood. 93, 1253-1263.

33. Istkovitz-Eldor, J., Schuldiner, M., Karsenti, D., Eden, A., Yanuka, O., Amit, M., Soreq, H., and Benvenisty, N. (2000). Differentiation of human embryonic stem cells into embryoid bodies comprising the three embryonic germ layers. Mol. Med. 6, 88-95.

34. Jackson, K. A., Mi, T., and Goodell, M. A. (1999). Hematopoietic potential of stem cells isolated from murine skeletal muscle. Proc. Natl. Acad. Sci. U.S.A. 96, 14482-14486.

35. Jackson, K., Majka, S. M., Wang, H., Pocius, J., Hartley, C. J., Majesky, M. W., Entman, M. L., Michael, L. H., Hirshi, K. K., and Goodell, M. A. (2001). Regeneratoin of ischemic cardiac muscle and vascular endotherlium by adult stem cells. J. Clin. Invest. 107, 1-8.

36. Johansson, B. M. and Wiles, M. V. (1995). Evidence for involvement of activin A and bone morphogenitic protein 4 in mammalian mesoderm and hematopoietic development. Mol. Cell. Biol. 15, 141-151.

37. Johansson, C. B., Momma, S., Clarke, D. L., Risling, M., Lendahl, U., and Frisen, J. (1999). Identification of a neural stem cell in the adult mammalian central nervous system. Cell. 96, 25-34.

38. Kawasaki, H., Mizuseki, K., Nishikawa, S., Kaneko, S., Kuwana, Y., Nakanishi, S. I., and Sasai, Y. (2000). Induction of midbrain dopaminergic neurons from ES cells by stromal cell-derived inducing activity. Neuron. 28, 31-40.

39. Kehat, I., Kenyagin-Karsenti, D., Druckmann, M., Segev, H., Amit, M., Gepstein, A., Livne, E., Binah, O., Itskovitz-Eldor, J., and Gepstein, L. (2001). Human embryonic stem cells can differentiate into myocytes portraying cardiomyocytic structural and functional properties. J. Clin. Invest. (in press).

40. Kerr, D.A., Llado, J., Shamblott, M., Maragakis, N., Irani, D. N., Dike, S., Sappington, A., Gearhart, J., and Rothstein, J. (2001). Human embryonic germ cell derivatives facilitate motor recovery of rats with diffuse motor neuron injury.

41. Klug, M. G., Soonpaa, M. H., Koh, G. Y., and Field, L. J. (1996). Genetically selected cardiomyocytes from differentiating embryonic stem cells form stable intracardiac grafts. J. Clin. Invest. 98, 216-224.

42. Kocher, A. A., Schuster, M. D., Szabolcs, M. J., Takuma, S., Burkhoff, D., Wang, J., Homma, s., Edwards, N. M, and Itescu, S. (2001). Neovascularization of ischemic myocardium by human bone-marrow-derived angioblasts prevents caardiomyocyte apoptosis, reduces remodeling and improves cardiac function. Nat. Med. 7, 430-436.

43. Kopen, G. C., Prockop, D. J., and Phinney, D. G. (1999). Marrow stromal cells migrate throughout forebrain and cerebellum, and they differentiate into astrocytes after injection into neonatal mouse brains. Proc. Natl. Acad. Sci. U.S.A. 96, 10711-10716.

44. Kramer, J., Hegert, C., Guan, K., Wobus, A. M., Muller, P. K., and Rohwedel, J. (2000). Embryonic stem cell-derived chondrogenic differentiation in vitro: activation by BMP-2 and BMP-4. Mech. Dev. 92, 193-205.

45. Krause, D. S., Theise, N. D., Collector, M. I., Henegariu, O., Hwang, S., Gardner, R., Neutzel, S., and Sharkis, S. J. (2001). Multi-organ, multi-lineage engraftment by a single bone marrow-derived stem cell. Cell. 105, 369-377.

46. Kuznetsov, S. A., Mankani, M. H., Gronthos, S., Satomura, K., Bianco, P., and Robey P. G. (2001). Circulating skeletal stem cells. J. Cell. Biol. 153, 1133-40.

47. Lagasse, E., Connors, H., Al Dhalirny, M., Reitsma, M., Dohse, M., Osborne, L., Wang, X., Finegold, M., Weissman, I. L. and Grompe, M. (2000). Purified hematopoietic stem cells can differentiate into hepatocytes in vivo. Nat. Med. 6, 1229-1234.

48. Lee, S. H., Lumeisky, N., Studer, L., Auerbach, J. M., and McKay, r. D. (2000). Efficient generation of midbrain and hindbrain neurons from mouse embryonic stem cells. Nat. Biotechnol. 18, 675-679.

49. Li, M., Pevny, L., Lovell-Badge, R., and Smith, A. (1998). Generation of purified neural precursors from embryonic stem cells by lineage selection. Curr. Biol. 8, 971-974.

50. Liechty, K. W., MacKenzie, T. C., Shaaban, A. F., Radu, A., Moseley, A. B., Dans, R, Marshak, D. r., and Flake, A. W. (2000). Human mesenchymal stem cells engraft and demonstrate site-specific differentiation after in utero transplantation in sheep. Nat. Med. 6, 1282-1286.

50. Liechty, K. W., MacKenzie, T. C., Shaaban, A. F., Radu, A., Moseley, A. B., Dans, R, Marshak, D. r., and Flake, A. W. (2000). Human mesenchymal stem cells engraft and demonstrate site-specific differentiation after in utero transplantation in sheep. Nat. Med. 6, 1282-1286.

51. Lieschke, G. J, and Dunn, A. R. (1995). Development of functional macrophages from embryonal stem cells in vitro. Exp. Heatol. 23, 328-334.

52. Liu, S., Qu, Y., Stewart, T. J., Howard, M. J., Chakrabortty, S., Holekamp, T. F., and Mcdonald, J. W. (2000). Embryonic stem cells differentiate into oligodendrocytes and myelinate in culture and after spinal cord transplantation. Proc. Natl. Acad. Sci. U.S.A. 97, 6126-6131.

53. Lumelsky, N., Biondel, O., Laeng, P., Velasco, I., Ravin, R., and McKay, R. (2001). Differentiation of Embryonic Stem Cells to Insulin-Secreting Structures Similar to Pancreatic Islets. Science. 292, 1389-1394.

54. Makino, S., Fukada, K., Miyoshi, S., Konishi, F., Kodama, H., Pan, J., Sano, M., Takahashi, T., Hori, S., Abe, H., Hata, J., Umezawa, A., and Ogawa, S. (1999). Cardiomyocytes can be generated from marrow stromal cells in vitro. J. Clin. Invest. 103, 697-705.

55. Maltsev, V. A., rohwedel, J., Hescheler, J., and Wobus, A. M. (1993). Embryonic stem cells differentiate in vitro into cardiomyocytes representing sinusnodal, atrial and ventricular cell types. Mech. Dev. 44, 41-50.

56. Matsui, Y., Zsebo, K., and Hogan, B. L. (1992). Derivation of pluripotential embryonic stem cells from murine primordial germ cells in culture. Cell. 70, 841-847.

57. McBurney, M. W., Reuhl, K. R., Ally, A. I., Nasipuri, S., Bell, J. C., and Craig, J. (1998). Differentiation and maturation of embryonal carcinoma-derived neurons in cell culture. J. Neurosci. 8, 1063-1073.

58. McCune, J. M., Namikawa, R., Kaneshima, H., Shultz, L. D., Lieberman, M., and Weissman, I. L. (1988). The SCID-hu mouse: murine model for the analysis of human hematolymphoid differentiation and function. Science. 241, 1632-1639.

59. Megeney, L. A., Kablar, B., Garrett, K., Anderson, J. E., and Rudnicki, M. A. (1996). MyoD is required for myogenic stem cell function in adult skeletal muscle. Genes Dev. 10, 1173-1183.

60. Mezey, E., Chandross, K. J., Harta, G., Maki, r. A., and McKercher, S. R. (2000). Turning blood into brain: cells bearing neuronal antigens generated in vivo from bone marrow. Science. 290, 1779-1782.

61. Morrison, S. J., Uchida, N., and Weissman, I. L. (1995). The biology of hematopoietic stem cells. Anuu. Rev. Cell. Dev. Biol. 11, 35-71.

62. namikawa, R., Weilbaecher, K. N., Kaneshima, H., Yee, E. J., and McCune, J. M. (1990). Long-term human hematopoieses in the SCID-hu mouse. J. Exp. Med. 172, 1055-1063.

63. O'Shea, K. S. (1999). Embryonic stem cell models of development. Anat. Rec. 257, 32-41.

64. Orlic, D., Kajstura, J., Chimenti, S., Jakoniuk, I., Anderson, S. M., Li, B., Pickel, J., McKay, R., Nadal-ginard, B., Bodine, D. M., Leri, A., and Anversa, P. (2001). Bone marrow cells regenerate infarcted myocardium. Nature. 410, 701-705.

65. Palmer, T. D., Schwartz, P. H., Taupin, P., Kaspar, B., Stein, S. A., and Gage, F. H. (2001). Progenitor cells from human brain after death. Nature. 411, 42-43.

66. Pera, M. F., Cooper, S., Mills, J., and Parrington, J. M. (1989). Isolation and characterization of a multipotent clone of human embryonal carcinoma cells. Differentiation. 42, 10-23.

67. Pera, M. F., and Herszfeld, D. (1998). Differentiation of human pluripotent teratocarcinoma stem cells induced by bone morphogenetic protein-2. Reprod. Fertil. Dev. 10, 551-555.

68. Pereira, R. F., Halford, K. W., O'Hara, M. D., Leeper, D. B., Sokolov, B. P., Pollard, M. D., Bagasra, O., and Prockop, D. J. (1995). Cultured adherent cells from marrow can serve as long-lasting precursor cells for bone, cartilage, and lung in irradiated mice. Proc. Natl. Acad. Sci. U.S.A. 92, 4857-4861.

69. Perkins, A. C. (1998). Enrichment of blood from embryonic stem cells in vitro. Reprod. Fertil. Dev. 10, 563-572.

70. Petersen, B. E., Bowen, W. C., Patrene, K. D., Mars, W. M., Sullivan, A. K., Murase, N.,Boggs, S. S., Greenberger, J. S., and Goff, J. P. (1999). Bone marrow as a potential source of hepatic oval cells. Science. 284, 1168-1170.

71. Pittenger, M. F., Mackay, A. M., Beck, S. C., Jaiswal, R. K., Douglas, r., Mosca, J. D., Moorman, M. A., Simonetti, D. W., Craig, S., and Marshak, D. R. (1999). Multilineage potential of adult human mesenchymal stem cells. Science. 284, 143-147.

72. Potocnik, A. J., Nielsen, P. J., and Eichmann, K. (1994). In vitro generation of lymphoid precursors from embryonic stem cells. EMBO. J. 13, 5274-5283.

73. Prelle, K., Wobus, A. M., Krebs, O., Blum, W. F., and Wolf, E. (2000). Overexpression of insulin-like growth factor-II in mouse embryonic stem cells promotes myogenic differentiation. Biochem. Biophys. Res. Commun. 277, 631-638.

74. Prockop, D. J. (1997). Marrow stromal cells as stem cells for nonhematopoietic tissues. Science. 276, 71-74.

75. Ramiya, V. K., Maraist, M., Arfors,K. E., Schatz, D. A., Peck, A. B., and Cornelius, J. g. (2000). Reversal of insulin-dependent diabetes using islets generated in vitro from pancreatic stem cells. Nat. Med. 6, 278-282.

76. Rathjen, P. D., Lake, J., Whyatt, L. M., Bettess, M. d., and Rathjen, J. (1998). Properties and uses of embryonic stem cells: prospects for application to human biology and gene therapy. Reprod. Fertil. Dev. 10, 31-47.

77. Reubinoff, B. E., Pera, M. F., Fong, C. Y., Trounson, A., and Bongso. A. (2000). Embryonic stem cell lines from human blastocysts: somatic differentiation in vitro. Nat. Biotechnol. 18, 399-404.

78. Reynolds, B. A., and Weiss, S. (1996). Clonal and population analyses demonstrate that an EGF-responsive mammalian embryonic CNS precursor is a stem cell. Dev. Biol. 175, 1-13.

79. Reynolds, J. N., Ryan, P. J., Prasad, A., and Paterno, G. D. (1994). Neurons derived from embryonal carcinoma (P19) cells express multiple GABA(A) receptor subunits and fully functional GABA(A) receptors. Neurosci. Lett. 165, 129-132.

80. Risau, W., Sarioloa, H., Zerwes, H. G., Sasse, J., Ekblom, P., Kemler, R., and Doetschman, T. (1988). Vasculogenesis and angiogenesis in embryhonic-stem-cell-derived embryoid bodies. Development. 102, 471-478.

81. Roach, S., Schmid, W., and Pera, M. F. (1994). Hepatocytic transcription factor expression in human embryonal carcinoma and yolk sac carcinoma cell lines: expression of HNF-3α in models of early endodermal cell differentiation. Exp. Cell. Res. 215, 189-198.

82. Rohwedel, J., Maltsev, V., Bober, E., Arnold, J. J., Hescheler, J., and Wobus, A. M. (1994). Muscle cell differentiation of embryonic stem cells reflects myogenesis in vivo: developmentally regulated expression of myogenic determination genes and functional expression of ionic currents. Dev. Biol. 164, 87-101.

83. Sanchez-Ramos, J., Song, S., Cardozo-Pelaez, F., Hazzi, C., Stedeford, T., Willing, A., Freeman, T. B., Saporta, S., Janssen, W., Patel, N., Cooper, D. R., and Sanberg, P. R. (2000). Adult bone marrow stromal cells differentiate into neural cells in vitro. Exp. Nerol. 164, 247-256.

84. Schuldiner, M., Yanuka, O., Itskovitz-Eldor, J., Melton, D., and Benvenisty, N. (2000). Effects of eight growth factors on the differentiatoin of cells derived from human embryonic stem cells. Proc. Natl. Acad. Sci. U.S.A. 97, 11307-11312.

85. Seale, P., Sabourin, L. A., Girgis-Gabardo, A., mansouri, A., Gruss, P., and Rudnicki, M. A. (2000). Pax7 is required for the specification of myogenic satellite cells. Cell. 102, 777-786.

86. Shamblott, M. J., Axelman, J., Littlefield, J. W., Blumenthal, P. D., Huggins, G. R., Cui, Y., Cheng, L., and Gearhart, J. D. (2001). Human embryonic germ cell derivatives express a broad range of developmentally distinct markers and proliferate extensively in vitro. Proc. Natl. Acad. Sci. U.S.A. 98, 12-118.

87. Shamblott, M. J., Axelman, J., Wang, S., Bugg, E. M., Littlefield, J. W., Donovan, P. J., Blumenthal, P. D., Huggins, G. R., and Gearhart, J. D. (1998). Derivation of pluripotent stem cells from cultured human primordial germ cells. Proc. Natl. Acad. Sci. U.S.A. 95, 13726-13731.

88. Slager, H. G., Van Inzen, W., Freund, E., Van den Eijnden-Van Raaij A. J. M., and Mummery, C. L. (1993). Transforming growth factor-beta in the early mouse embryo: implications for the regulation of muscle formation and implantation. Dev. Genet. 14, 212-224.

89. Soria, B., Roche, E., Berna, G., Leon-Quinto, T., Reig, J. A., and Martin, F. (2000). Insulin-secreting cells derived from embryonic stem cells normalize glycemia in streptozotocin-induced diabetic mice. Diabetes. 49, 157-162.

90. Spangrude, G. J., Smith,, L., Uchida, N., Ikuta, K., Heimfeld, S., Friedman, J., and Weissman, I. L. (1991). Mouse hematopoietic stem cells. Blood. 78, 1395-1402.

91. Strubing, C., Ahnert-Hilger, G., Shan, J., Wiedenmann, B., Hescheler, J., and Wobus, A. M. (1995). Differentiation of pluripotent embryonic stem cells into the neuronal lineage in vitro gives rise to mature inhibitory and exitatory neurons. Mech. Dev. 53, 275-287.

92. Taniguchi, H.,Toyoshima, T., Fukao, K., and Nakauchi, H. (1996). Presence of hematopoietic stem cells in the adult liver. Nat. Med. 2, 198-203.

93. Theise, N. D., Nimmakayalu, M., Gardner, R., Illei, P. B., Morgan, G., Teperman, L., Henegarlu, O., and Krause, D. S. (2000). Liver from bone marrow in humans. Hepatology. 32, 11-16.

94. Thompson, S., Stem, P. L., Webb, M., Walsh, F. S., Engstrom, W., Evans, E. P., Whi, W. K., Hopkins, B., and Graham, C. F. (1984). Cloned human teratoma cells differentiate into neuron-like cells and other cell types in retinoic acid. J. Cell. Sci. 72, 37-64.

95. Thomson, J. A., Itskovitz-Eldor, J., Shapiro, S. S., Waknitz, M. A., Swiergiel, J. J., Marshall, V. S., and Jones, J. M. (1998). Embryonic stem cell lines derived from human blastocysts. Science. 282, 1145-1147.

96. Tomita, S., Li, R. K., Weisel, R. D., Mickle, D. A., Kim, E. J., Sakai, T., and Jia, Z. Q. (1999). Autologous transplantation of bone marrow cells improves damaged heart function 672. Circulation. 100(Suppl. II), 11247-11256.

97. Trojanowski, J. Q., Mantione, J. R., Lee, J. H., Seid, D. P., You, T., Inge, L. J., and Lee, V. M. (1993). Neurons derived from a human teratocarcinoma cell line establish molecular and structural polarity following transplantation into the rodent brain. Exp. Neurol. 122, 283-294.

98. Tsai, M., Wedemeyer, J., Ganiatsas, S., Tam, S. Y., Zon, L. I., and Galli, S. J. (2000). In vivo immunological function of mast cells derived from embryonic stem cells: an approach for the rapid analysis of even embryonic lethal mutations in adult mice in vivo. Proc. Natl. Acad. Sci. U.S.A. 97, 9186-9190.

99. Uchida, N., Buck, D. W., He, D., Reitsma, M. J., Masek, M., Phan, T. V., Tsukamoto, A. S., Gage, F. H., and Weissman, I. L. (2000). Direct isolation of human central nervous system stem cells. Proc. Natl. Acad. Sci. U.S.A. 97, 14720-14725.

100. Wakitani, S., Saito, T., and Capian, A. I. (1995). Myogenic cells derived from rate bone marrow mesenchymal stem cells exposed to 5-azacytidine 754. Muscle. Nerve. 18, 1417-1426.

101. Wang, X., Al-Dhalimy, M., Lagasse, E., Finegold, M., and Grompe, M. (2001). Liver repopulation and correction of metabolic liver disease by transplanted adult mouse pancreatic cells. Am. J. Pathol. 158, 571-579.

102. Weiss, S., Dunne, C., Hewson, J., Wohl, C., Wheatley, M., Peterson, A. C., and Reynolds, B. A. (1996). Multipotent CNS stem cells are present in the adult mammalian spinal cord and ventricular neuroaxis. J. Neurosci. 16, 7599-7609.

103. Westfall, M. V., Pasyk, K. A., Yule, D. I., Samuelson, L. C., and Metzger, J. M. (1997). Ultrastructure and cell-cell coupling of cardiac myocytes differentiating in embryonic stem cell cultures. Cell. Motil. Cytoskeleton. 36, 43-54.

104. Wiles, M. v. and Keller, G. (1991). Multiple hematopoietic lineages develop from embryonic stem (ES) cells in culture. Development. 111, 259-267.

105. Wobus, A. M., Rohwedel, J., Maltsev, V., and Hescheler, J. (1995). Development of cardiomyocytes expressing cardiac-specific genes, action potentials, and ionic channels during embryonic stem cell-derived cardiogenesis. Ann. N.Y. Acad. Sci. 752, 460-469.

106. Woodbury, D., Schwarz, E. J., Prockop, D. J., and Black, I.B. (2000). Adult rat and human bone marrow stromal cells differentiate into neurons. J. Neurosci. Res. 61, 364-370.

107. Yamane, T., Hayashi, H., Mizoguchi, M., Yamazaki, H., and Kunisada, T. (1999). Derivation of melanocytes from embryonic stem cells in culture. Dev. Dyn. 216, 450-458.

108. Yamashita, J., Itoh, H., Hirashima, M., Ogawa, M., Nishikawa, S., Yurugi, T., Naito, M., Nakao, K., and Nishikawa, S. (2000). Flk1-positive cells derived from embryonic stem cells serve as vascular progenitors. Nature. 408, 92-96.

109. Zuk, P. A., Zhu, M., Mizuno, H., Huang, J., Futrell, J. W., Katz, A. J., Benhaim, P., Lorenz, H. P., and Hedrick, M. H. (2001). Multilineage cells from human adipose tissue: implications for cell-based therapies. Tissue Eng. 7, 211-228.

110. Zulewski, H., Abraham, E. J., Berlach, M. J., Deniel, P. B., Moritz, W., Muller, B., Vellejo, M., Thomas, M K., and Habener, J. F. (2001). Multipotential nestin-positive stem cells isolated from adult pancreatic islets differentiate ex vivo into pancreatic endocrine, exocrine, and hepatic phenotypes. Diabetes. 50, 521-533.

Applications of PNES Cells and Advantages of PNES Cells Relative to other Pluripotent Cell Lines

Scientific and therapeutic applications of the technology and composition of this invention include, but are not limited to, the following:

1. Studies on human development and the origin of the disease. Help understand complexities of formation of human organs and tissues. Most major diseases are due to abnormal cell specialization and cell division. PNES cells give us a key research tool for understanding fundamental events in human development, such as explaining the causes of birth defects, and approaches to prevent or correct.

2. Drug discovery, drug evaluation, drug testing and drug development. To test a drug or chemical's efficacy or toxicity, the scientific community currently uses animal models in vitro using cells from rats, mice and other animals, or in vivo tests that involve giving the drug or chemical to the animal to test safety. Beside the ethical considerations, these tests/models are not always predictive for what will happen in human beings. Human models to date usually involve established cell lines that have been maintained in vitro for a long period of time. These cell lines are usually transformed and differ significantly from primary cells in vivo, making these established cell lines of limited utility. PNES cells can help in overcoming many if not all of these shortcomings.

3. Treatment of diseases and disorders including, but not limited to, Parkinson's, Alzehimer's, Huntington's, Ty Sachs, Gauchers, spinal cord injury, stoke, burns and other skin damage, heart disease, diabetes, Lupus, osteoarthritis, liver diseases, hormone disorders, kidney disease, leukemia, lymphoma, multiple sclerosis, rheumatoid arthritis, Duchenne's Musclar Dystrophy, Ontogenesis Imperfecto, birth defects, infertility, pregnancy loss, and other cancers, degenerative and other diseases and disorders.

4. Genomics/Gene Manipulation/Delivery Devices. Scientists predict that human stem cells such as PNES will be useful vehicles for delivering genes to specific tissues. The current alternative, viral delivery devices, have significant limitations (e.g., some viruses only attack dividing cells, not all cells, so application is limited, and there are risks of harmful immune reaction associated with this mechanism). PNES cells can offer a more robust delivery system that can overcome these limitations.

In addition to providing these promising applications, PNES cells also have characteristics and properties that make them a more attractive alternative when compared with ES cell lines created under current technologies. These advantages include, but are not limited to, the following.

1. The creation of PNES cells doesn't involve embryos (naturally created or created via cloning), fetal tissue or the mixing of species.

2. Current ES cell lines come from a limited genetic pool whereas PNES cell lines can be created from an unlimited genetic pool and can be created specifically for a given patient or patient population (e.g., PNES can be autologous) and thus PNES cells avoid another likely barrier to the use of ES cell lines—immune rejection.

3. PNES cell lines can be created on an ongoing basis, whereas because of certain limitations imposed by the NIH and proposed legislation, the creation of new ES cell lines for human is under severe scrutiny and faces significant barriers. The ES cell lines that currently exist and are approved for federally funded applications will likely be subject to genetic changes and mutations as they age, e.g., they can't be kept healthy in culture indefinitely.

4. PNES cells for humans can be created and proliferated in cultures without using mouse feeding layers, so as to avoid the mixing of species.

Applications of Invention's ASC's and Specific Differentiated Cells and Advantages Over other Sources:

The application of the ASC's and Specific Differentiated Cells created by the current invention include, but not exclusively,

1. Drug discovery, drug evaluation, drug testing and drug development. To test a drug or chemical's efficacy or toxicity, the scientific community currently uses animal models in vitro using cells from rats, mice and other animals, or in vivo tests that involve giving the drug or chemical to the animal to test safety. Beside the ethical considerations, these tests/models are not always predictive for what will happen in human beings. Human models to date usually involve established cell lines that have been maintained in vitro for a long period of time. These cell lines are usually transformed and differ significantly from primary cells in vivo, making these established cell lines of limited utility. ASC's and Specific Differentiated Cells, which the current invention can produce on an ongoing basis including multiple cell lines, can help in overcoming many if not all of these shortcomings.

2. Treatment of diseases and disorders including, but not limited to, Parkinson's, Alzehimer's, Huntington's, Ty Sachs, Gauchers, spinal cord injury, stoke, burns and other skin damage, heart disease, diabetes, Lupus, osteoarthritis, liver diseases, hormone disorders, kidney disease, leukemia, lymphoma, multiple sclerosis, rheumatoid arthritis, Duchenne's Musclar Dystrophy, Ontogenesis Imperfecto, birth defects, infertility, pregnancy loss, and other cancers, degenerative and other diseases and disorders

3. Genomics/Gene Manipulation/Delivery Devices. Scientists predict that human stem cells such as PNES will be useful vehicles for delivering genes to specific tissues. The current alternative, viral delivery devices, have significant limitations (e.g., some viruses only attack dividing cells, not all cells, so application is limited, and there are risks of harmful immune reaction associated with this mechanism). PNES cells can offer a more robust delivery system that can overcome these limitations.

In addition to providing these promising applications, ASC's and Specific Differentiated Cells produced under the current invention have characteristics and properties that make them a more attractive alternative when compared with multipotent/adult stem cells produced or secured from other sources (such as in vivo, umbilical cords and other limited sources):

1. Under the current invention, ASC's and Specific Differentiated Cells can be produced without the use and destruction of embryos (naturally created or created via cloning), fetal tissue or the mixing of species.

2. Current methods for producing multipotent/adult stem cells and Specific Differentiated Cells (derived from current ES lines) utilize a limited genetic pool whereas multipotent ASC's and Specific Differentiated Cells produced under the current invention can be created from an unlimited genetic pool and can be created specifically for a given patient or patient population (e.g., cells produced under this invention can be autologous) and thus this invention's source for these cells avoids another likely barrier to the use of these cells derived from ES cell lines—immune rejection.

3. This invention can create multipotent ASC's and Specific Differentiated Cells on an ongoing basis, whereas because of certain limitations imposed by the NIH and proposed legislation, the creation of new ES cell lines for humans and derivatives thereof including multipotent and undifferentiated cells is under severe scrutiny and faces significant barriers, and the current ES cell lines and derivatives thereof will likely be subject to problems as they age such as genetic changes and mutations—e.g., they can't be kept healthy in culture indefinitely.

4. ASC's from in vivo sources have not been identified for all human tissues whereas PNES have the ability to differentiate into cells derived from all three embryonic germ layers.

5. In vivo sourced ASC's and Specific Differentiated Cells are in short supply and costly to accumulate or harvest. The current invention offers a more efficient and productive source.

6. In vivo ASC's are much more difficult to isolate than ASC's created under the current invention.

### Detailed Description of the Preferred Embodiments

In order that this invention may be better understood, the following examples are set forth. These examples are for the purpose of illustration only and are not to be construed as limiting the scope of the invention in any manner.

### EXAMPLE I

Maturation of Bovine Oocytes

Bovine methaphase II oocytes were obtained from a commercial source (Ovagenix, San Angelo, Tex.). The supplier obtained immature oocytes from a slaughterhouse source. Immature oocytes were washed in HEPES buffered embryo culture medium (HECM supplemented with 10% FCS). Next, the supplier placed immature oocytes into maturation medium consisting of tissue culture medium (TCM) 199 containing 10% fetal calf serum which contains appropriate gonadotropins such as luteinizing hormone (LH) and follicle stimulating hormone (FSH), and estradiol. The commercial supplier then placed the maturing oocytes in a battery powered portable incubator, and shipped the incubator via overnight mail to arrive in our laboratory the next morning. Therefore the maturation period occurred while the oocytes were in transit. The maturation period is defined as period beginning from the time of introducing the immature oocytes into the maturation medium until the time at which the mature oocytes are utilized in the present study. The current invention utilizes bovine mature metaphase II oocytes with a 18 to 36 hour maturation period. Mature metaphase II bovine oocytes were washed in HECM. Unwanted granulosa cells were removed from the oocytes by treatment consisting of incubating the cells in a solution of 0.5-1.0 mg/ml hyaluronidase (Sigma H3757) followed by mechanical pipetting of the cells using a fine bore Pasteur pipette. Next, the denuded oocytes were washed in HECM prior to micromanipulation to remove any hyaluronidase residue. Only mature Metaphase II bovine oocytes of normal quality were utilized further in this procedure.

### EXAMPLE II

Micromanipulation and Enucleation of Bovine Oocytes

Micromanipulation and enucleation of bovine oocytes was performed as follows. Micromanipulation was performed on a inverted microscope (Nikon, Japan) using micromanipulators (Narashige, Japan). The mature metaphase II oocytes were introduced to HECM containing 10% Plasmanate and 7.5-15.0 μg/ml cytochalasin B (Sigma C6762). Next, a holding micropipette (Humagen, Charlottesville, Va.) was used to grasp the oocytes. While holding the oocyte, the zona pellucida of each oocyte was partially dissected (dissolved) by application of an acidic tyrodes solution (Sigma T1788). The acidic tyrodes solution was applied using a 20-30 μm diameter micropipette (Humagen, Charlottesville, Va.). The zona was dissolved adjacent to the polar body of the mature oocyte. Following breach of the zona, a 20-50 μm micrometer polished micropipette (Humagen, Charlottesville, Va.) was used to gently aspirate the polar body and underlying cytoplasm, which was pinched away from the remaining ooplasm. This procedure was repeated for each oocyte. The resulting “enucleated” oocytes and the removed polar body and underlying ooplasm were stained using 5 μg/ml Hoechst 33342 (Sigma) and microscopically viewed briefly (<10 seconds) using ultraviolet irradiation to confirm that all nuclear DNA has been removed from the enucleated oocytes. Only successfully enucleated oocytes were utilized further.

### EXAMPLE III

Ooplastoid Generation from Bovine Oocytes

Ooplastoid generation for bovine oocytes was performed as follows. Enucleated oocytes were introduced to HECM containing 10% Plasmanate and 7.5-15.0 μg/ml cytochalasin B. A micromanipulator (Narashige, Japan) was used to manipulate the enucleated oocytes. A holding micropipette (Humagen 10MPH-120, Charlottesville, Va.) was used to grasp and orient the enucleated oocytes. A 20-50 μm polished micropipette (Humagen custom, Charlottesville, Va.) was used to gently aspirate and pinch off a portion of the enucleated oocyte. This process was repeated until each enucleated oocyte was partitioned into 3-5 zona pellucida free ooplastoids having from 20 to 33% of the volume of the original oocyte. This procedure was repeated until each enucleated oocyte was appropriately partitioned into ooplastoids. Ooplastoids were washed in HECM with 10% Plasmanate to remove Cytochalasin B for further micromanipulation.

### EXAMPLE IV

Preparation of Bovine Somatic Cells for Nuclear Transfer

The source of bovine somatic cell nucleus for experiments described here has been granulosa cells. Granulosa cells were obtained from bovine oocyte/granulosa masses. The granulosa masses were subjected to chemical treatment with 0.5-1.0 mg/ml hyaluronidase (Sigma H3757) followed by mechanical removal of granulosa through repeated pipetting of the cells using fine bore Pasteur pipettes. Subsequently, the isolated granulosa cells were washed with HECM with 10% Plasmanate to remove hyaluronidase. Next, granulosa were cultured in ECM or HECM supplemented with 10% FCS or Plasmanate in preparation for further micromanipulation. Alternatively, granulosa or any other type of somatic cell may be cultured in ECM supplemented with 0.5% fetal calf serum or Plasmanate for 24 to 72 h to induce quiescence prior to nuclear transfer.

### EXAMPLE V

Nuclear Transfer of Somatic Cell Nuclei to Bovine Ooplastoids Using Electrofusion and Creation of Nascent Cells/P-PNES

No Nuclear transfer of bovine somatic cell nuclei to ooplastoids was performed by cell electrofusion. For bovine ooplastoids electrofusion was performed as follows. Micromanipulation of ooplastoids and granulosa was performed using a micromanipulator (Narashige, Japan). A 10-20 μm polished micropipette was used to aspirate a single granulosa cell. The granulosa cell was positioned firmly against the plasma membrane of a single ooplastoid, using mechanical pressure to maximize cell-to-cell contact. During this step the HECM may be supplemented with 100-200 μg/ml Phytohaemagglutinin to improve cell-to-cell contact. This procedure was repeated for each ooplastoid resulting in the formation of ooplastoid/somatic cell aggregates or pairs.

The ooplastoid/somatic cell aggregates were then very gently aspirated and moved to a fusion chamber (BTX) containing fusion medium (0.3 M mannitol, 0.1 mM MgSO₄, 0.05 mM CaCl₂). Next, using an electroporator, model (BTX 2001) two DC pulses of 0.1-2.0 kilovolts/cm and 25 μs were applied to the fusion chamber to induce cell fusion. After electroporation the ooplastoid/somatic cell aggregates were gently removed from the fusion chamber and incubated in ECM with 20% Plasmanate or FCS. Cell fusion was visually confirmed or denied 20-30 minutes post electroporation by observation using an inverted microscope (Nikon, Japan). Successfully fused pairs were referred to as P-PNES or “nascent cells.” The P-PNES were moved to a 30 mm Petri dish (Nunc, Denmark) containing culture medium (Quinns Advantage Cleavage Medium, Sage Biopharma, Bedminster, N.J.) supplemented with 10% Plasmanate or FCS and cultured in 6% CO2. P-PNES were observed for cleavage division over the next 72 h.

### EXAMPLE VI

Activation of Bovine Ooplastoids, or P-PNES Cells

Activation of bovine oocytes, ooplastoids, or nascent cells is a specific procedure that may be applied at one or more times during the overall laboratory process described here. Activation may be mechanical (simply pricking the cell with a fine bore needle or injection pipette), electrical (applying a DC pulse as in electrofusion), or chemical (calcium ionophore or ethanol treatment). Activation may be applied to the mature oocyte prior to the micromanipulation procedures. Depending on the species and conditions, activation may be achieved during enucleation of the oocyte, ooplastoid partitioning, or during intracytoplasmic injection of the somatic cell nucleus. Activation may also be achieved during the application of the DC pulse during the electrofusion process. In the current invention bovine P-PNES cells were activated as a result of electrofusion DC pulse with acceptable levels of activation achieved in each case. Alternatively, the frequency of successful activation may be enhanced by adding a pre or post micromanipulation activation step if improvements are desired for this critical process.

### EXAMPLE VII

Superovulation and Collection of Mouse Oocytes

Murine (mouse) oocytes were obtained by inducing superovulation of 4-8 week old females (B6CBA/F1, Jackson Lab) by first administering intraperitoneal (IP) injections of 5 IU Pregnant Mare Serum Gonadotropin, (Calbiochem 367222) and 5 IU of hCG (Sigma). Next, the mice were sacrificed at 22 h post hCG injection and the ovaries and fallopian tubes were dissected to remove oocytes. The recovered oocytes were then washed in HECM (Conception Technologies, EH500) supplemented with 10% Plasmanate (Bayer, Elkhart, Ind.). Granulosa cells were removed from the oocyte preparation by treatment of 0.5-1.0 mg/ml hyaluronidase (Sigma H3757) followed by mechanical pipetting of the cells using a fine bore Pasteur pipette. The denuded oocytes were washed in HECM prior to micromanipulation to remove hyaluronidase residue. Only mature metaphase II mouse oocytes were utilized further in this procedure.

### EXAMPLE VIII

Micromanipulation and Enucleation of Mouse Oocytes

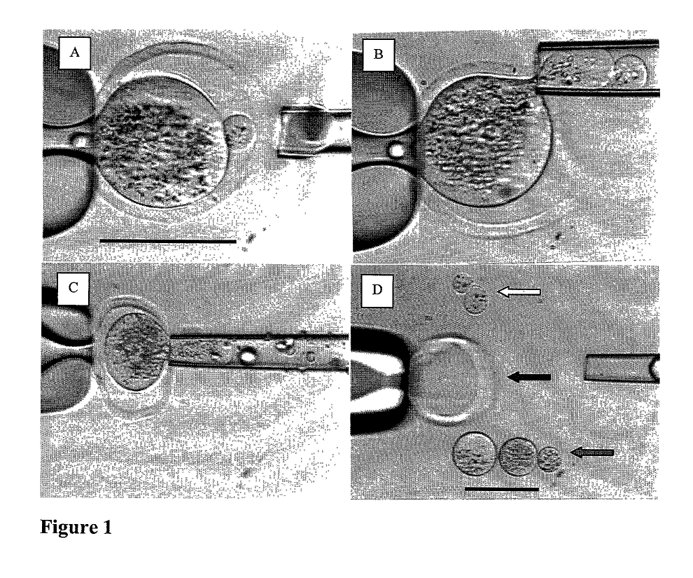

Micromanipulation and enucleation of mouse oocytes was performed as follows. Micromanipulation was performed on a inverted microscope (Nikon, Japan) using micromanipulators (Narashige, Japan). The MII Mature oocytes were introduced to HECM containing 10% Plasmanate and 7.5-15.0 μg/l cytochalasin B (Sigma C6762). Next, a holding micropipette (Humagen, Charlottesville, Va.) was used to grasp the oocytes (FIG. 1A). While holding the oocyte, the zona pellucida of each oocyte was partially dissected (dissolved) by application of an acidic tyrodes solution (Sigma T1788). The acidic tyrodes solution was applied using a 20-30 μm diameter micropipette (Humagen, Charlottesville, Va.). The zona was dissolved adjacent to the polar body of the mature oocyte. Following breach of the zona a 20-50 μm micrometer polished micropipette (Humagen, Charlottesville, Va.) was used to gently aspirate the polar body and underlying cytoplasm, which was pinched away from the remaining ooplasm (FIG. 1B). This procedure was repeated for each oocyte. The resulting “enucleated” oocytes and the removed polar body and underlying ooplasm was stained using 5 μg/ml Hoechst 33342 (Sigma) and viewed briefly (<10 seconds) using ultraviolet irradiation to confirm that all nuclear DNA has been removed from the enucleated oocytes. Only successfully enucleated oocytes were utilized further.

### EXAMPLE IX

Ooplastoid Generation from Mouse Oocytes

Ooplastoid generation for mouse oocytes was performed as follows. Enucleated oocytes were introduced to HECM containing 10% Plasmanate and 7.5-15.0 μg/ml Cytochalasin B. A micromanipulator (Narashige, Japan) was used to manipulate the enucleated oocytes. A holding micropipette (Humagen 10MPH-120, Charlottesville, Va.) was used to grasp and orient the enucleated oocytes. A 20-50 μm polished micropipette (Humagen custom, Charlottesville, Va.) was used to gently aspirate and pinch off a portion of the enucleated oocyte (FIG. 1C). This process was repeated until each enucleated oocyte was partitioned into 2-6 zona pellucida-free ooplastoids having from about 17% to less than 50% of the volume of the original oocyte (FIG. 1D). This procedure was repeated until each enucleated oocyte was appropriately partitioned into ooplastoids. Ooplastoids were washed in HECM with 10% Plasmanate to remove Cytochalasin B for further micromanipulation.

### EXAMPLE X

Preparation of Mouse Somatic Cells for Nuclear Transfer

The source of mouse somatic cell nucleus for experiments described here has been granulosa cells. Granulosa cells were obtained from mouse oocyte/granulosa masses. The granulosa masses were subjected to chemical treatment with 0.5-1.0 mg/ml hyaluronidase (Sigma H3757) followed by mechanical removal of granulosa through repeated pipetting of the cells using fine bore Pasteur pipettes. Subsequently, the isolated granulosa cells were washed with HECM with 10% Plasmanate to remove hyaluronidase. Next, granulosa were cultured in ECM or HECM supplemented with 10% Plasmanate in preparation for further micromanipulation. Alternatively, granulosa or any other type of somatic cell may be cultured in ECM supplemented with 0.5% fetal calf serum or Plasmanate for 24 to 72 h to induce quiescence prior to nuclear transfer.

### EXAMPLE XI

Nuclear Transfer of Somatic Cell Nucleus by Direct Intracytoplasmic Injection

Nuclear transfer of mouse somatic cell nucleus to the ooplastoids may be achieved by cell fusion or by direct intracytoplasmic injection. Direct injection of granulosa nuclei into mouse ooplastoids was performed as follows. Micromanipulation of ooplastoids and granulosa was performed using a micromanipulator (Narashige, Japan). A blunt or pointed injection micropipette with a 8-15 μm diameter, slightly smaller than the granulosa cell, was used to pick up one granulosa cell. The granulosa cell was repeatedly aspirated and expelled from the pipette in order to break the cell membrane. The granulosa cell was immediately injected into a single ooplastoid, which was gently grasped by a holding pipette. The medium used for this micromanipulation was HECM with 10% Plasmanate and may be supplemented with 7.5-15.0 μg/ml Cytochalasin B to minimize cell lysis. This procedure was repeated for each ooplastoid. Each successfully injected ooplastoid containing a single granulosa cell nucleus is referred to as a P-PNES. The P-PNES were moved to a 30 mm Petri dish (Nunc, Denmark) containing culture medium (Quinns Advantage Cleavage Medium, Sage Biopharma, Bedminster, N.J.) supplemented with 10% Plasmanate or FCS and cultured in 6% CO2. P-PNES were observed for cleavage division over about the next 72-96 h.

### EXAMPLE XII

Activation of Mouse Oocytes, Ooplastoids, and P-PNES Cells

Activation of oocytes, ooplastoids or P-PNES cells is a specific procedure that may be applied at one or more times during the overall laboratory process described here. Activation may be mechanical (simply pricking the cell with a fine bore needle or injection pipette), electrical (applying a DC pulse as in electrofusion), or chemical (calcium ionophore or ethanol treatment). Activation may be applied to the mature oocyte prior to the micromanipulation procedures. Depending on the species and conditions, activation may be achieved during enucleation of the oocyte, ooplastoid partitioning, or during intracytoplasmic injection of the somatic cell nucleus. Activation may also be achieved during the application of the DC pulse during the electrofusion process. In the current invention a portion of the mouse ooplastoids or P-PNES cells were activated as a result of intracytoplasmic nucleus injection. The frequency of successful activation of mouse ooplastoids P-PNES cells was enhanced by adding a post micromanipulation activation step consisting of electroporation. This involved moving the P-PNES cells to a fusion chamber (BTX) containing fusion medium (0.3 M mannitol, 0.1 mM MgSO₄, 0.05 mM CaCl₂). Next, using an electroporator, model (BTX 2001) two DC pulses of 0.1-2.0 kv/cm and 25 μs were applied to the electroporation.

### EXAMPLE XIII

Culture of Human, Mouse, and Bovine PNES or P-PNES Cells and Prevention of Cell Clumping

P-PNES/nascent cells of all species produced by somatic cell nuclear transfer are cultured in ECM (Quinns Advantage Cleavage Medium, Sage Biopharma, Bedminster, N.J.) supplemented with 10% Plasmanate(Bayer), HSA, or FCS at 5-6% CO2 at 37° C. Each P-PNES/nascent cell in this invention is cultured individually for about 72 to about 96 h. P-PNES cells are observed using an inverted Nikon Eclipse microscope with a heated (37° C.) stage at about 24, 48, 72, and 96 h post micromanipulation/activation. In the human, mouse, and bovine each P-PNES/nascent cell will cleave (divide mitotically) to form about two to four separate cells at about 24 h post activation, fabout our to eight separate cells at about 48 h post activation, and about eight or more cells at about 72 to 96 h. Dividing cells at about 72 to 96 h post activation begin to form plasma membrane contact between adjacent cells. To prevent formation of cell to cell membrane connections, the cells are separated by mechanical (pipetting) treatment and chemical treatment with hyaluronidase, trypsin, chymotrypsin or similar chemical treatment in calcium and magnesium free phosphate buffered saline with 10% FCS. Mechanically separated cells originating from different P-PNES/nascent cells are pooled at about 72 to 96 h post activation. Pooled P-PNES cells all originated from the same somatic cell donor/source are presumed autologous to each other as well as the somatic cell donor/source.

### EXAMPLE XIV

Culture of Human, Mouse, and Bovine P-PNES Cells for Formation of PNES Cells

For human, mouse, and bovine cells, 100 to 200 pooled P-PNES cells at about 72 to 96 h post activation are introduced to a fibroblast feeder culture system as follows. For culture human, mouse, and bovine P-PNES cells mouse fetal fibroblasts are isolated from postcoitum fetuses. Mitomicin or ultra-violet inactivated fibroblasts are cultured in monolayers at 70,000 to 90,000 cells/cm² in Nunc 35×10 mm culture dishes, in DMEM supplemented with 10% FCS, L.I.F., and S.I.T. (Sigma), with 5-6% CO2 at 37° C. Alternatively, for culture of human P-PNES cells at about 72 to 96 h post activation, human fibroblast monolayers may be substituted. The source of the human fibroblasts used for the continuous PNES culture ideally is autologous to the source of the somatic cell used for nuclear transfer, however screened donor fibroblast cultures may be substituted.

Disaggregated, pooled P-PNES cells at about 72 to 96 hour post activation are introduced and spread upon the inactivated fibroblast monolayer using a sterile Pasteur pipette. Cells are observed periodically for the about next 48 h and mechanically disaggregated using a Pasteur pipette if clumps of cells are observed. This is repeated until cells are observed to adhere to the feeder layer. On about day 3 to 7 after introducing the cells to the feeder layer the cell colonies are observed for mechanical cell sorting. Cells on the monolayer are manipulated using an inverted microscope equipped with a micromanipulator and a polished 25 μm micropipette. Alternatively, a hand drawn sterile Pasteur pipette is used to mechanically manipulate cultured cells while the technician is viewing the cell colonies with a stereomicroscope. Cells exhibiting embryonic stem cell like morphology as defined by Thompson (U.S. Pat. No. 6,200,806) are selected and physically separated from the monolayer and aspirated into a micropipette or Pasteur pipette. The selected cells are then transferred (passaged) to a new inactivated fibroblast feeder layer for continued culture. As mentioned above, these cells are referred to as pluripotent non-embryonic stem cells or PNES. PNES cells are passaged to a new inactivated fibroblast monolayer culture about every 7 to 10 days according to standard embryonic stem cell culture techniques.

### EXAMPLE XV

Analysis of PNES Cells

Aliquots of these human, mouse, and bovine PNES cells are characterized as stem cells using the stem cell markers. For human PNES cells are SSEA-1(−).SSEA-3(+).SSEA-4(+).TRA-1-60(+).TRA-1-81(+). The cells are tested using immunofluorescent microscopy. The mouse monoclonal antibodies to stage-specific embryonic antigens (SSEA) 1.3 and 4 are available from Hybridoma Bank at NIH. TRA-1-60 and TRA-1-80 are available from Vector Laboratories. To certify PNES cells for the presence or absence of the indicated markers, the cells are placed on the cover slips on an irradiated mouse embryonic fibrolasts (3000 rad) allowed them to adhere and spread, and fixed with 4% formalin. Following fixation and staining with different antibodies the presence of the marker is identified by binding the FITC labeled rabbi anti-mouse polyclonal antibodies. As a control the embryocarcinoma (EC) cell line NTERA-2 cl. D1 (available from ATCC) are used.

### EXAMPLE XVI

Method for Constructing Super-Ooplasts that Are Greater than the Size of a Normal Oocyte

Ooplasts may theoretically be of any size or volume. In contrast to constructing ooplast that are by volume smaller than an oocyte, ooplasts may be constructed that are actually larger than a normal oocyte. To create large ooplasts, several oocytes of any mammalians species are enucleated in HECM containing 10% FCS and about 7.5-15.0 μg/ml Cytochalasin B (Sigma C6762) using micromanipulation techniques as previously described. The zona pellucida of all enucleated oocytes is removed using mechanical action or using chemical agents. The enucleated oocytes (ooplasts) are then introduced into a fusion chamber containing a fusion medium such as 0.3 M mannitol, 0.1 mM MgSO₄, 0.05 mM CaCl₂. Within the fusion chamber two or more ooplasts are aligned with membrane-to-membrane contact in an axis perpendicular to the electrodes. Using an electroporator one or more electrical pulses are applied with defined parameters such as 0.1-2.0 kilovolts/cm, 25 μs/pulse. After applying the pulse the ooplasts may fuse to form a non-nucleated super-ooplast consisting of a volume greater than one normal oocye. This may be repeated to form super-ooplasts of theoretically any volume.

It is contemplated that the invention includes methods of producing and utilizing PNES cells and their and their derivatives, i.e., Specific Differentiated Cells including, but not limited to sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. in scientific and therapeutic applications including, but not limited to, (a) scientific discovery and research involving cellular development and genetic research, (b) drug development and discovery (e.g., screening for efficacy and toxicity of certain drug candidates and chemicals), (c) gene therapy (e.g., as a delivery device for gene therapy), and (d) treatment of diseases and disorders including, but not limited to, Parkinson's, Alzehimer's, Huntington's, Ty Sachs, Gauchers, spinal cord injury, stoke, burns and other skin damage, heart disease, diabetes, Lupus, osteoarthritis, liver diseases, hormone disorders, kidney disease, leukemia, lymphoma, multiple sclerosis, rheumatoid arthritis, Duchenne's Musclar Dystrophy, Ontogenesis Imperfecto, birth defects, infertility, pregnancy loss, and other cancers, degenerative and other diseases and disorders.

While we have hereinbefore described a number of embodiments of this invention, it is apparent that our basic constructions can be altered to provide other embodiments that utilize the processes and compositions of this invention. Therefore, it will be appreciated that the scope of this invention is to be defined by the claims appended hereto rather than by the specific embodiments that have been presented hereinbefore by way of example.

1. A purified preparation of pluripotent non-embryonic stem cells, which
(i) is capable of proliferating in an in vitro culture for more than one year;
(ii) maintains a karyotype in which the cells are euploid and are not altered through culture;
(iii) maintains the potential to differentiate into cell types derived from the endoderm, mesoderm and ectoderm lineages throughout the culture, and
(iv) is inhibited from differentiation when cultured on fibroblast feeder layers.

2. The pluripotent non-embryonic stem cells of claim 1, wherein said cells are negative for expression of the SSEA-1 marker.

3. The pluripotent non-embryonic stem cells of claim 1, wherein said cells express elevated alkaline phosphatase activity.

4. The pluripotent non-embryonic stem cells of claim 1, wherein said cells are positive for expression of the TRA-1-81 marker and the TRA-1-60 marker.

5. The pluripotent non-embryonic stem cells of claim 1, wherein said cells are positive for expression of the CCA-3 and CCA-4 Markers.

6. The pluripotent non-embryonic stem cells of claim 1, wherein said cells differentiate into cells derived from mesoderm, endoderm and ectoderm germ layers when the cells are injected into a SCID mouse.

7. The pluripotent non-embryonic stem cells of claim 1, wherein said cells are human.

8. The pluripotent non-embryonic stem cells of claim 1, wherein said cells are non-human animal cells selected from the group consisting of dog, cat, mouse, rat, cow, pig, sheep, goat, horse, buffalo, llama, ferret, guinea pig and rabbit.

9. The pluripotent non-embryonic stem cells of claim 1, wherein the nuclear DNA has been genetically modified.

10. A purified preparation of pluripotent non-embryonic stem cells, which
(i) is capable of proliferating in an in vitro culture for an indefinite period;
(ii) maintains a karyotype in which the cells are euploid and are not altered through culture; and
(iii) maintains the potential to differentiate into cells types derived from the endoderm, mesoderm and ectoderm lineages throughout the culture.

11. The pluripotent non-embryonic stem cells of claim 10, wherein said cells are negative for expression of the SSEA-1 marker.

12. The pluripotent non-embryonic stem cells of claim 10, wherein said cells express elevated alkaline phosphatase activity.

13. The pluripotent non-embryonic stem cells of claim 10, wherein said cells are positive for expression of the TRA-1-8 1 marker and the TRA-1-60 marker.

14. The pluripotent non-embryonic stem cells of claim 10, wherein said cells are positive for expression of the CCA-3 and CCA-4 Markers.

15. The pluripotent non-embryonic stem cells of claim 10, wherein said cells differentiate into cells derived from mesoderm, endoderm and ectoderm germ layers when the cells are injected into a SCID mouse.

16. The pluripotent non-embryonic stem cells of claim 10, wherein said cells are human.

17. The pluripotent non-embryonic stem cells of claim 10, wherein said cells are non-human animal cells selected from the group consisting of dog, cat, mouse, rat, cow, pig, sheep, goat, horse, buffalo, llama, ferret, guinea pig and rabbit.

18. The pluripotent non-embryonic stem cells of claim 10, wherein the nuclear DNA has been genetically modified.

19. A stem cell which does not originate from a fertilized egg, but which originates from the combination of a somatic cell nucleus and an enucleated ooplastoid.

20. The stem cells of claim 19, wherein said cells are negative for expression of the SSEA-1 marker.

21. The stem cells of claim 19, wherein said cells express elevated alkaline phosphatase activity.

22. The stem cells of claim 19, wherein said cells are positive for expression of the TRA-1-81 marker and the TRA-1-60 marker.

23. The stem cells of claim 19, wherein said cells are positive for expression of the CCA-3 and CCA-4 Markers.

24. The stem cells of claim 19, wherein said cells differentiate into cells derived from mesoderm, endoderm and ectoderm germ layers when the cells are injected into a SCID mouse.

25. The stem cells of claim 19, wherein said cells are human.

26. The stem cells of claim 19, wherein said cells are non-human animal cells selected from the group consisting of dog, cat, mouse, rat, cow, pig, sheep, goat, horse, buffalo, llama, ferret, guinea pig and rabbit.

27. The stem cells of claim 19, wherein the nuclear DNA has been genetically modified.

28. The stem cells of claim 19, wherein said enucleated ooplastoid comprises less than the cytoplasmic volume of the original egg from which it is derived.

29. The stem cells of claim 19, wherein said enucleated ooplastoid comprises from about 10% to about 100% of the cytoplasmic volume of the original egg from which it is derived.

30. A stem cell which is produced by the method of (i) contacting a desired somatic cell or somatic cell nucleus with an ooplastoid, wherein said ooplastoid is derived from an enucleated oocyte; (ii) combining said somatic cell or somatic cell nucleus with said ooplastoid to create a nascent cell, and (iii) culturing said nascent cell to obtain pluripotent non-embryonic stem cells.

31. The stem cells of claim 30, wherein said cells are negative for expression of the SSEA-1 marker.

32. The stem cells of claim 30, wherein said cells express elevated alkaline phosphatase activity.

33. The stem cells of claim 30, wherein said cells are positive for expression of the TRA-1-81 marker and the TRA-1-60 marker.

34. The stem cells of claim 30, wherein said cells are positive for expression of the CCA-3 and CCA-4 Markers.

35. The stem cells of claim 30, wherein said cells differentiate into cells derived from mesoderm, endoderm and ectoderm germ layers when the cells are injected into a SCID mouse.

36. The stem cells of claim 30, wherein said cells are human.

37. The stem cells of claim 30, wherein said cells are non-human animal cells selected from the group consisting of dog, cat, mouse, rat, cow, pig, sheep, goat, horse, buffalo, llama, ferret, guinea pig and rabbit.

38. The stem cells of claim 30, wherein the nuclear DNA has been genetically modified.

39. The stem cells of claim 30, wherein said enucleated ooplastoid comprises less than the cytoplasmic volume of the original egg from which it is derived.

40. The stem cells of claim 30, wherein said enucleated ooplastoid comprises from about 10% to about 100% of the cytoplasmic volume of the original egg from which it is derived.

41. A nascent cell produced from the combination of a somatic cell nucleus and an enucleated zona pellucida free ooplastoid.

42. The nascent cell of claim 41, which is activated by a series of electrical pulses.

43. The nascent cell of claim 41, which is activated by the addition of a chemical activator.

44. The nascent cell of claim 41, which is activated by the addition of a chemical activator selected from the group consisting of ethanol, inositol trisphosphate, calcium ionophores, strontium ions, 6-dimethylaminopurine, cyclohexamide, and phorbol 12-myristate 13-acetate.

45. The nascent cell of claim 41, having from about 10% to about 100% of the cytoplasmic volume of the original egg from which it is derived.

46. The nascent cell of claim 41, having less than 50% of the cytoplasmic volume of the original egg from which it is derived.

47. A method of producing pluripotent, non-embryonic stem cells comprising the following steps:
(i) contacting a desired somatic cell or somatic cell nucleus with an ooplastoid, wherein said ooplastoid is derived from an enucleated oocyte;
(ii) combining said somatic cell or somatic cell nucleus with said ooplastoid to create a nascent cell;
(iii) activating said nascent cell; and
(iv) culturing said nascent cell to obtain pluripotent non-embryonic stem cells.

48. The method according to claim 47, wherein said somatic cell or somatic cell nucleus is a mature cell.

49. The method according to claim 47, wherein said somatic cell is an epithelial cell, lymphocyte or fibroblast.

50. The method according to claim 47, wherein said combining step involves intracytoplasmic injection of the somatic cell nucleus into the zona free reduced volume ooplastoid.

51. The method according to claim 47, wherein said combining step involves fusion in an electric field via electroporation.

52. The method according to claim 47, wherein said combining step involves fusion induced by electrodes that are introduced directly into the culture dish and electrical pulses administered to the couplets immediately following micromanipulation.

53. The method according to claim 47, wherein said combining step involves fusion in a fusion chamber.

54. The method according to claim 47, wherein said ooplastoid contains less than 50% of the cytoplasmic volume of a mature oocyte.

55. The method according to claim 47, wherein said ooplastoid contains from about 10% to about 100% of the cytoplasmic volume of a mature oocyte.

56. A cell line obtained according to the method of claim 47.

57. A method of producing pluripotent non-embryonic stem cells comprising the following steps:
(i) contacting one or more desired somatic cells or somatic cell nuclei with a super-ooplast derived from one or more enucleated zona pellucida free oocytes;
(ii) dividing said super-ooplast into single nucleus containing nascent cells;
(iii) activating said nascent cells; and
(iv) culturing said nascent cells to obtain pluripotent non-embryonic stem cells.

58. The method according to claim 57, wherein said enucleated zona pellucida free super-ooplast comprises more than 100% of the cytoplasmic volume of a single egg.

59. The method according to claim 57, wherein said somatic cell or somatic cell nucleus is a mature cell.

60. The method according to claim 57, wherein said somatic cell is an epithelial cell, lymphocyte or fibroblast.

61. The method according to claim 57, wherein said dividing step involves partitioning said super-ooplast into separate single nuclei containing nascent cells.

62. The method according to claim 57, wherein said contacting step involves intracytoplasmic injection of said somatic cell nucleus into said super-ooplast.

63. The method according to claim 57, wherein said activation step involves fusion in an electric field via electroporation.

64. The method according to claim 57, wherein said activation step involves fusion in a fusion chamber.

65. The method according to claim 57, wherein said activation step involves fusion induced by electrodes that are introduced directly into the culture dish and electrical pulses administered to the couplets immediately following micromanipulation.

66. The method according to claim 57, wherein said nascent cell is activated using electrical pulses.

67. The method according to claim 57, wherein said nascent cell is activated during a fusion process.

68. A cell line obtained according to the method of claim 57.

69. A method of producing an ooplastoid comprising the following steps:
(i) harvesting an oocyte from a female;
(ii) maturing said oocyte to metaphase II;
(iii) breaching or removing the zona pelucida of said metaphase II oocyte;
(iv) enucleating said oocyte by removing the polar body and nuclear DNA of said oocyte through the breach of the zona pelucida or by oocyte partitioning; and
(v) aspirating and pinching off an ooplastoid from said enucleated oocyte.

70. The method of claim 69, wherein said oocyte is from a human.

71. The method of claim 69, wherein said oocyte is from a non-human animal selected from the group consisting of dog, cat, mouse, rat, cow, pig, sheep, goat, horse, buffalo, llama, ferret, guinea pig and rabbit.

72. The method of claim 69, wherein said zona pelucida is breached or removed using a chemical agent.

73. The method of claim 69, wherein said zona pelucida is breached or removed using mechanical action.

74. The method of claim 69, wherein said ooplastoid has from about 10% to about 100% of the volume from the original oocyte.

75. The method of claim 69, wherein said ooplastoid has from about 15% to about 49% of the volume from the original oocyte.

76. The method of claim 69, wherein said ooplastoid has from about 17% to about 33% of the volume from the original oocyte.
